(19) United States
(12) Patent Application Publication
(10) Pub. No.: US 2008/0267910 A1
(43) Pub. Date: Oct. 30, 2008

(54) Rat, rabbit, and cynomolgus monkey IL-21 and IL-22 nucleotide and polypeptide sequences

(76) Inventor: Paul W. Wu, Cambridge, MA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/073,707

(22) Filed: Mar. 7, 2008

Related U.S. Application Data
(60) Provisional application No. 60/905,836, filed on Mar. 9, 2007.

(51) Int. Cl. A61K 38/20; C07H 21/04; C12N 5/06; C12N 15/63; C07K 14/435; A61K 39/395; C07K 16/24; A61K 38/02; C07K 14/54; C12P 21/02; G01N 33/566

(52) U.S. Cl. 424/85.2; 536/23.5; 435/325; 435/320.1; 530/350; 530/351; 435/69.52; 436/501; 514/2; 424/130.1; 530/387.9

(57) ABSTRACT

This disclosure provides isolation and characterization of nucleotides encoding rat and cynomolgus monkey IL-21 and rat, rabbit and cynomolgus monkey IL-22, and the IL-21 and IL-22 polypeptides encoded thereby. This disclosure features rat and cynomolgus monkey IL-21 and rat, rabbit and cynomolgus monkey IL-22 nucleotide and amino acid sequences and variants thereof.

### RELATED APPLICATION INFORMATION

This application claims priority to provisional application 60/905,836, filed Mar. 9, 2007, which is incorporated by reference in its entirety.

### FIELD

This disclosure relates to discovery, identification and characterization of nucleotide and polypeptide sequences for rat and cynomolgus monkey IL-21 and IL-22 and rabbit IL-22.

### BACKGROUND

Cytokines are a large and diverse group of molecules that mediate proliferation, differentiation and survival of hematopoietic cells. They are usually produced de novo in response to an immune stimulus and generally act by binding to specific membrane receptors, which then signal the target cell via second messenger pathways. Cytokines may elicit different types of responses in target cells, including, for example, increasing or decreasing expression of various membrane proteins and receptors, proliferation properties of target cells and secretion of effector molecules.

Interleukin-21 (IL-21) is a class I cytokine which has been reported to play an important role in the regulation of natural killer (NK) and T cell functions. For example, IL-21 has been reported to play an important role in the proliferation of T cells and in affecting the cytolytic activity of NK cells. IL-21 has also been shown to up-regulate genes associated with innate immunity and to inhibit the differentiation of naive T helper cells. IL-21 specifically inhibits interferon-gamma (IFN-γ) production from developing Th1 cells and is believed to be preferentially expressed by Th2 cells. Furthermore IL-21 has been identified as a growth and survival factor for human myeloma cells, which suggests a potential therapeutic application of IL-21 in the treatment of cancers.

Interleukin-22 (IL-22) is a class II cytokine that shows sequence homology to IL-10. Its expression is up-regulated in T cells by IL-9 or ConA (Dumoutier L. et al. (2000) Proc Natl Acad Sci USA 97(18):10144-9)). Additionally, studies have shown that expression of IL-22 mRNA is induced in vivo in response to LPS administration, and that IL-22 modulates parameters indicative of an acute phase response (Dumoutier L. et al. (2000) supra; Pittman D. et al. (2001) Genes and Immunity 2:172)). In addition, IL-22 enhances the expression of antimicrobial peptides associated with host defense, including β-defensin, S100A7, S100A8, and S100A. Wolk et al., Immunity, 21:241-54 (2004); Boniface et al., J. Immunol. 174:3695-3702 (2005). Taken together, these observations indicate that IL-22 plays a role in inflammation (Kotenko S. V. (2002) Cytokine & Growth Factor Reviews 13(3):223-40)).

### SUMMARY

The present disclosure relates to the discovery, identification and characterization of polynucleotides derived from rat, rabbit and cynomolgus monkey and the encoded polypeptides of IL-21 and IL-22. The present disclosure further encompasses vectors, host cells, antibodies, and methods for producing these polypeptides. Also provided are diagnostic methods for detecting disorders associated with an increase or decrease in the level of IL-21 and/or IL-22, and therapeutic methods for treating such disorders. The disclosure also relates to screening methods for identifying agonists and antagonists of IL-21 and IL-22. These cytokines share structural and functional homology with their counterparts in other organisms.

In some embodiments, the present disclosure provides an isolated nucleic acid molecule comprising a nucleotide sequence as set forth in SEQ ID NO:1 or SEQ ID NO:3 or a variant thereof encoding a polypeptide having IL-21 activity. The present disclosure also comprises an isolated nucleic acid molecule comprising nucleotides 1 to 438 of SEQ ID NO:1 or nucleotides 1 to 486 of SEQ ID NO:3, which encode the open reading frames of the IL-21 polypeptides, or a variant thereof encoding a polypeptide having IL-21 activity. The present disclosure also comprises an isolated nucleic acid molecule comprising nucleotides 52 to 438 of SEQ ID NO:1 or nucleotides 88 to 486 of SEQ ID NO:3, which encode the predicted mature forms of the IL-21 polypeptides, or a variant thereof encoding a polypeptide having IL-21 activity. In other embodiments, the present disclosure provides an isolated nucleic acid molecule comprising a nucleotide sequence as set forth in SEQ ID NO:5, SEQ ID NO:7, or SEQ ID NO:39 or variant thereof encoding a polypeptide having IL-22 activity. The present disclosure also comprises an isolated nucleic acid molecule comprising nucleotides 1 to 537 of SEQ ID NO:5, nucleotides 32 to 568 of SEQ ID NO:7, or nucleotides 1 to 561 of SEQ ID NO:39 which encode the open reading frames of the IL-22 polypeptides, or a variant thereof encoding a polypeptide having IL-22 activity. The present disclosure also comprises an isolated nucleic acid molecule comprising nucleotides 100 to 537 of SEQ ID NO:5, nucleotides 131 to 568 of SEQ ID NO:7, or nucleotides 100 to 561 of SEQ ID NO:39 which encode the predicted mature forms of the IL-22 polypeptides, or a variant thereof encoding a polypeptide having IL-22 activity.

The present disclosure further encompasses polypeptides having IL-21 activity and comprising an amino acid sequence set forth in SEQ ID NO:2 or 4. The present disclosure also comprises an isolated polypeptide molecule comprising amino acids 18 to 146 of SEQ ID NO:2 or amino acids 30 to 162 of SEQ ID NO:4, which comprise the predicted mature forms of the IL-21 polypeptides, or a variant thereof comprising a polypeptide having IL-21 activity. The present disclosure also encompasses polypeptides having IL-22 activity and comprising an amino acid sequence set forth in SEQ ID NO:6, SEQ ID NO:8 or SEQ ID NO:40, or amino acids 34 to 179 of SEQ ID NO:6, amino acids 34 to 179 of SEQ ID NO:8, or amino acids 34 to 187 of SEQ ID NO:40 which correspond to the predicted mature form of the IL-22 polypeptides, or a variant thereof comprising a polypeptide having IL-22 activity.

In addition, the present disclosure also encompasses nucleotide sequences which encode the amino acid sequences set forth in SEQ ID NOs:2, 4, 6, 8, and 40 and amino acids 18 to 146 of SEQ ID NO:2, 30 to 162 of SEQ ID NO:4, 34 to 179 of SEQ ID NO:6, 34 to 179 of SEQ ID NO:8, 34 to 187 of SEQ ID NO:40, and variants of such sequences with either IL-21 or IL-22 activity.

This disclosure further provides vectors containing a DNA molecule containing a nucleotide sequence set forth in SEQ ID NO:1, 3, 5, 7, 39, nucleotides 1 to 438 of SEQ ID NO:1, 52 to 438 of SEQ ID NO:1, 1 to 486 of SEQ ID NO:3, 88 to 486 of SEQ ID NO:3, 1 to 537 of SEQ ID NO:5, 100 to 537 of SEQ ID NO:5, 32 to 568 of SEQ ID NO:7, 131 to 568 of SEQ ID NO:7, 1 to 561 of SEQ ID NO:39, 100 to 561 of SEQ ID NO:39, or a variant thereof. In certain embodiments, a nucleotide sequence set forth in SEQ ID NO:1, 3, 5, 7, 39, nucleotides 1 to 438 of SEQ ID NO:1, 52 to 438 of SEQ ID NO:1, 1 to 486 of SEQ ID NO:3, 88 to 486 of SEQ ID NO:3, 1 to 537 of SEQ ID NO:5, 100 to 537 of SEQ ID NO:5, 32 to 568 of SEQ ID NO:7, 131 to 568 of SEQ ID NO:7, 1 to 561 of SEQ ID NO:39, 100 to 561 of SEQ ID NO:39 or a variant thereof is operably linked to a heterologous promoter which expresses an IL-21 or IL-22 polypeptide or a variant thereof encoded by the nucleotide sequence.

This disclosure also provides host cells transfected with a vector containing a nucleotide sequence set forth in SEQ ID NO:1, 3, 5, 7, 39, nucleotides 1 to 438 of SEQ ID NO:1, 52 to 438 of SEQ ID NO:1, 1 to 486 of SEQ ID NO:3, 88 to 486 of SEQ ID NO:3, 1 to 537 of SEQ ID NO:5, 100 to 537 of SEQ ID NO:5, 32 to 568 of SEQ ID NO:7, 131 to 568 of SEQ ID NO:7, 1 to 561 of SEQ ID NO:39, 100 to 561 of SEQ ID NO:39 or a variant thereof. Examples of host cells include, but are not limited to, 293, CHO, COS, HEK, NSO, BaF3, and BHK.

Methods of producing polypeptides described herein are also provided. Such methods include, for example, culturing a cell transfected with a vector containing a DNA molecule comprising a nucleotide sequence set forth in SEQ ID NO:1, 3, 5, 7, 39, nucleotides 1 to 438 of SEQ ID NO:1, 52 to 438 of SEQ ID NO:1, 1 to 486 of SEQ ID NO:3, 88 to 486 of SEQ ID NO:3, 1 to 537 of SEQ ID NO:5, 100 to 537 of SEQ ID NO:5, 32 to 568 of SEQ ID NO:7, 131 to 568 of SEQ ID NO:7, 1 to 561 of SEQ ID NO:39, 100 to 561 of SEQ ID NO:39 or a variant thereof, operably linked to a heterologous promoter, and recovering the protein.

### BRIEF DESCRIPTION OF THE DRAWINGS

FIG. 1 shows an alignment between the nucleotide sequence of rat IL-21 (rIL-21) (SEQ ID NO:1) and a murine IL-21 sequence (mIL-21) (SEQ ID NO:9), demonstrating about 93% identity between nucleotide (nt) 1 to nt 438 of SEQ ID NO:1 and nt 1 to nt 438 of SEQ ID NO:9.

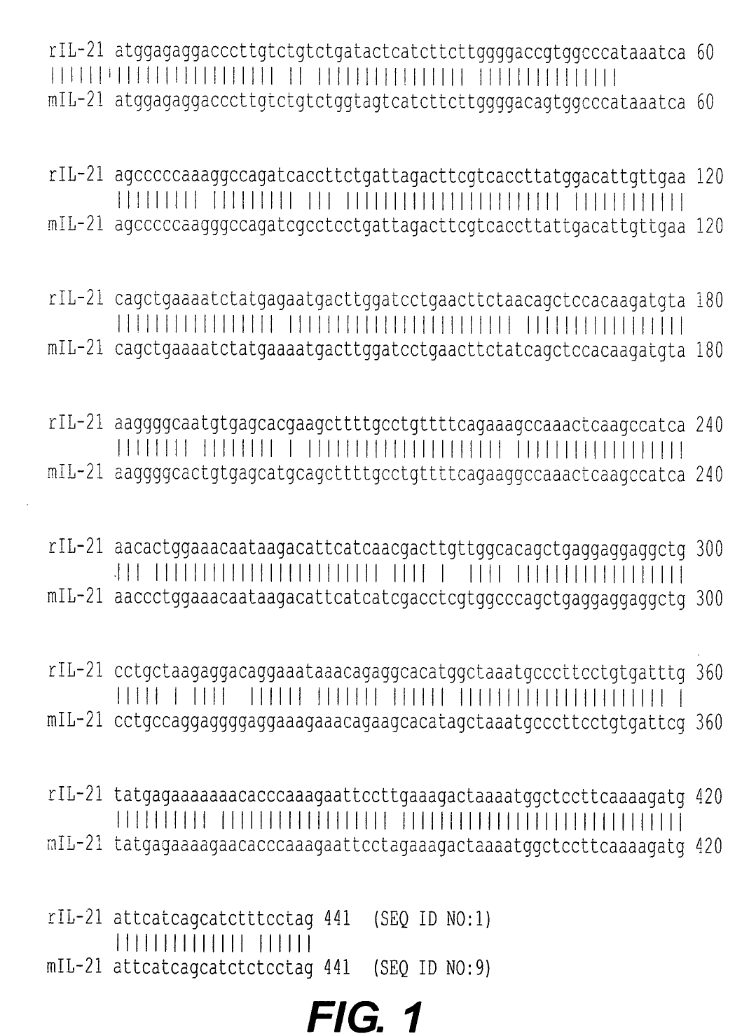

FIG. 2 shows an alignment between the amino acid sequence of rat IL-21 (SEQ ID NO:2) and a murine IL-21 sequence (SEQ ID NO:10), demonstrating about 87% identity between amino acid (aa) 1 to aa 146 of SEQ ID NO:2 and aa 1 to aa 146 of SEQ ID NO:10.

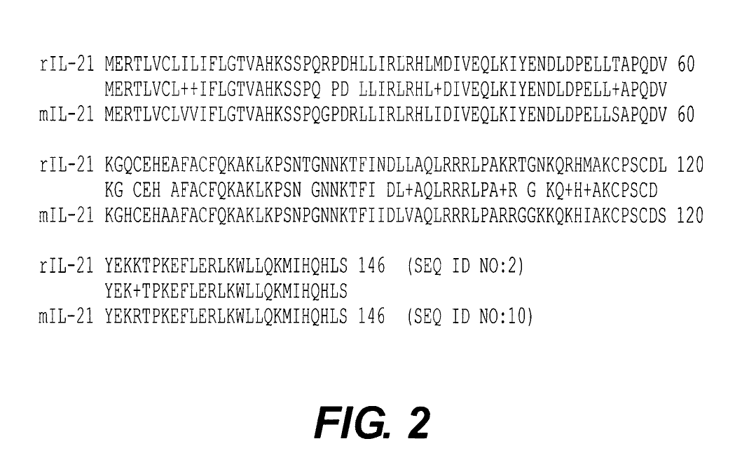

FIG. 3 shows an alignment between the nucleotide sequence of cynomolgus monkey IL-21 (cIL-21) (SEQ ID NO:3) and a human IL-21 (hIL-21) sequence (SEQ ID NO:11), demonstrating about 98% identity between nt 1 to nt 486 of SEQ ID NO:3 and nt 46 to nt 531 of SEQ ID NO:11.

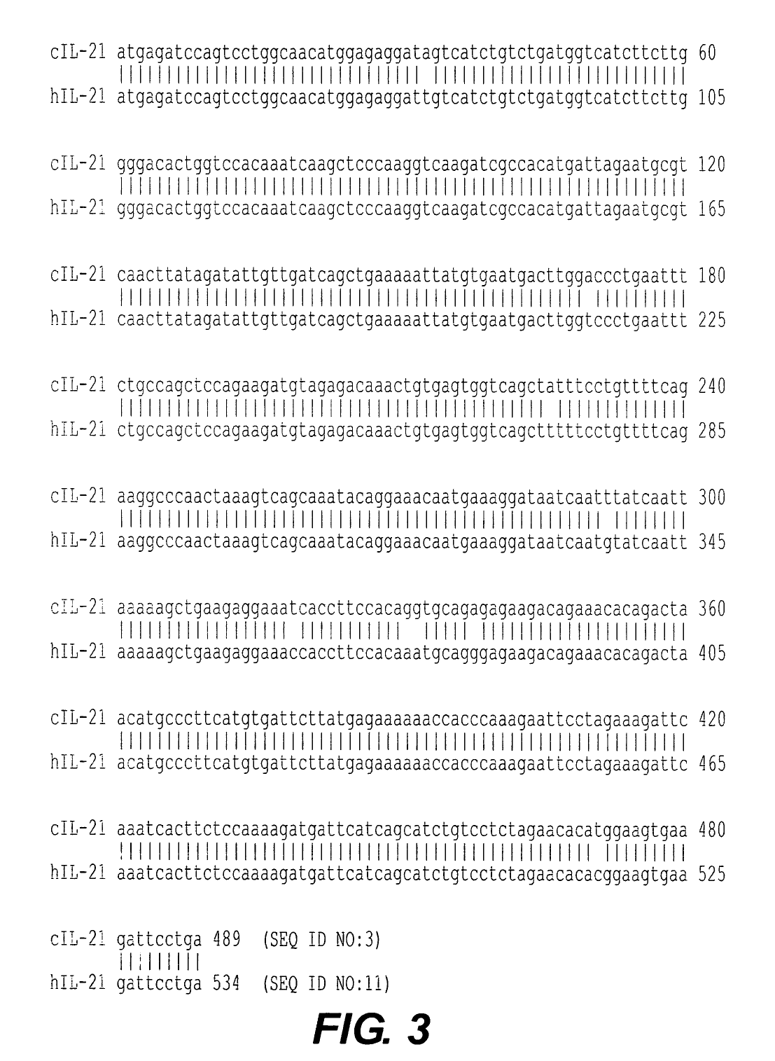

FIG. 4 shows an alignment between the amino acid sequence of cynomolgus monkey IL-21 (SEQ ID NO:4) and a human IL-21 sequence (SEQ ID NO:12), demonstrating about 96% identity between aa 1 to aa 162 of SEQ ID NO:4 and aa 1 to aa 162 of SEQ ID NO:12.

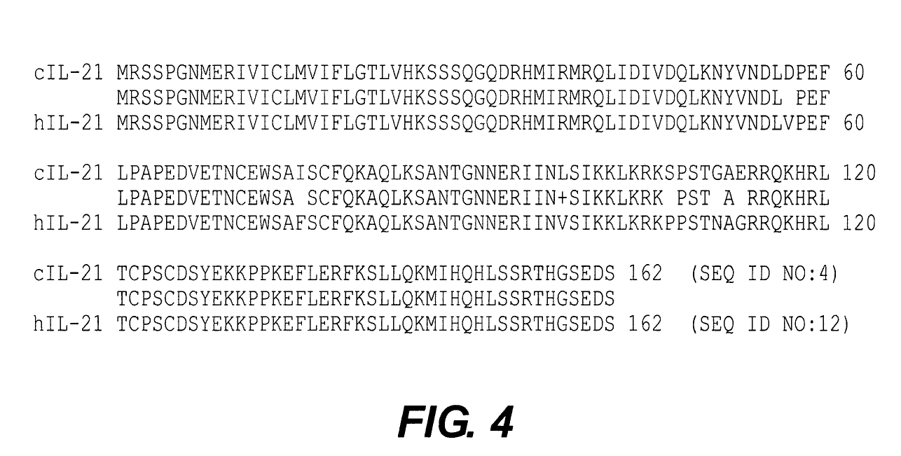

FIG. 5 shows an alignment between the nucleotide sequence of rat IL-22 (SEQ ID NO:5) and a murine IL-22 sequence (SEQ ID NO:13), demonstrating about 90% identity between nt 1 to nt 537 of SEQ ID NO:5 and nt 54 to nt 590 of SEQ ID NO: 13.

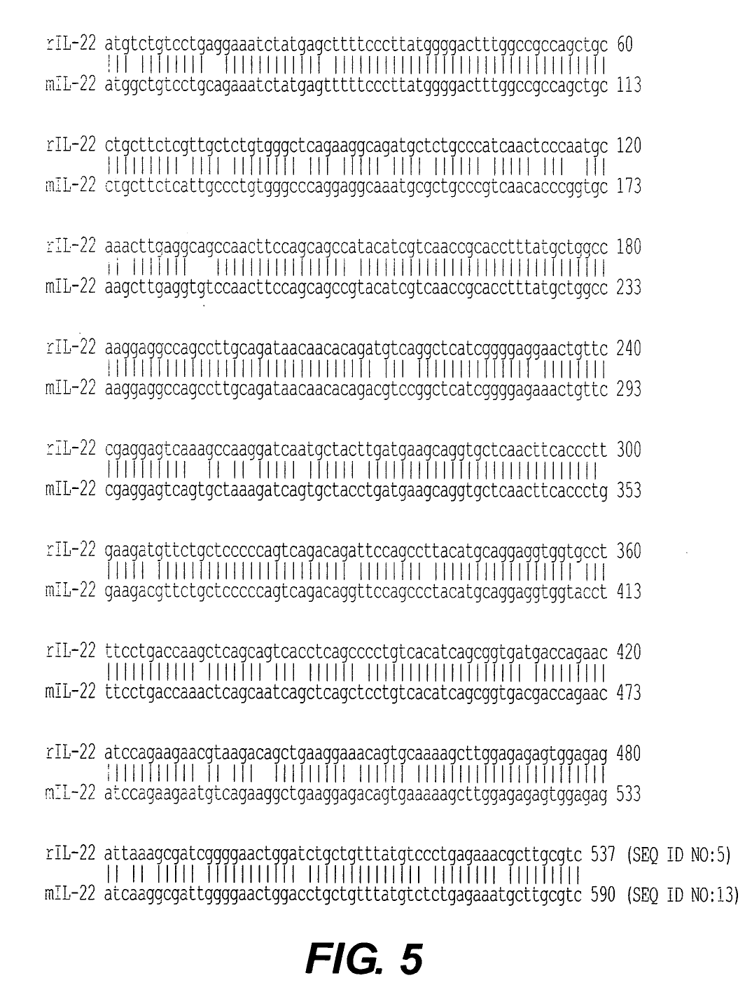

FIG. 6 shows an alignment between the amino acid sequence of rat IL-22 (SEQ ID NO:6) and a murine IL-22 sequence (SEQ ID NO:14), demonstrating about 90% identity between aa 1 to aa 179 of SEQ ID NO:6 and aa 1 to aa 179 of SEQ ID NO:14.

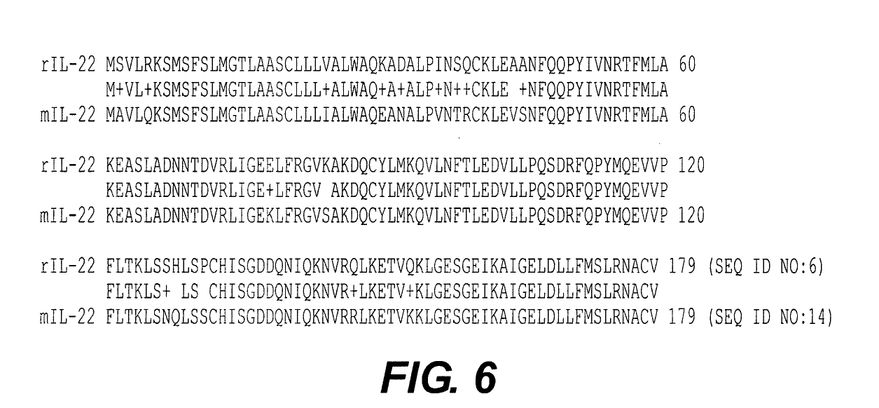

FIG. 7 shows an alignment between the nucleotide sequence of cynomolgus monkey IL-22 (SEQ ID NO:7) and a human IL-22 sequence (SEQ ID NO:15), demonstrating about 97% identity between nt 32 to nt 568 of SEQ ID NO:7 and nt 58 to nt 594 of SEQ ID NO:15.

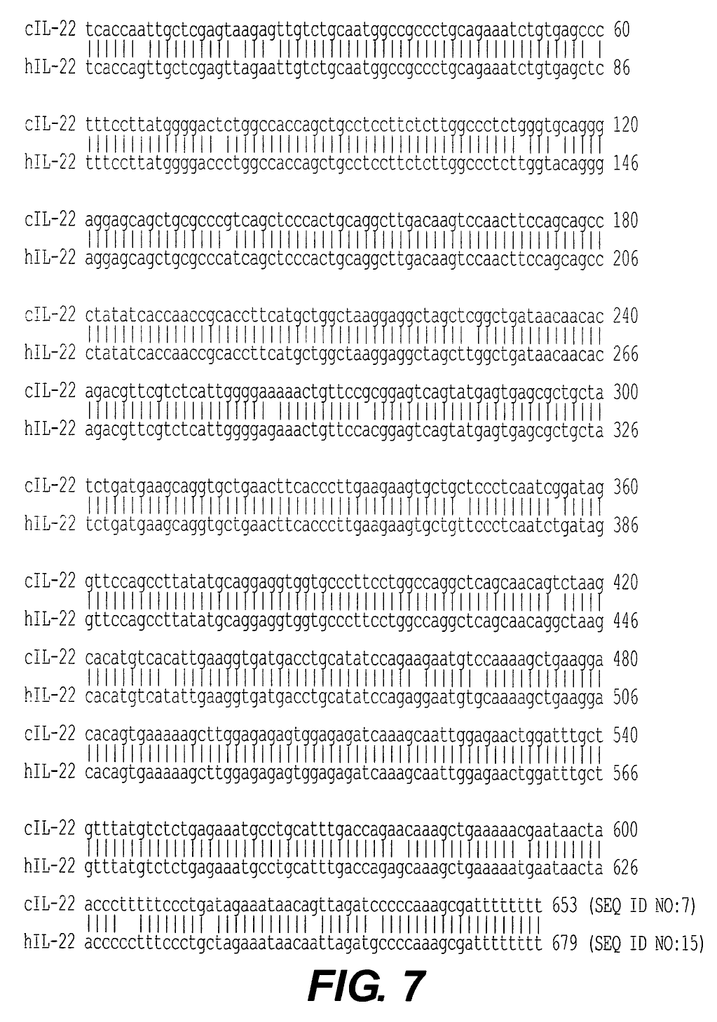

FIG. 8 shows an alignment between the amino acid sequence of cynomolgus monkey IL-22 (SEQ ID NO:8) and a human IL-22 sequence (SEQ ID NO:16), demonstrating about 96% identity between aa 1 to aa 179 of SEQ ID NO:8 and aa 1 to aa 179 of SEQ ID NO:16.

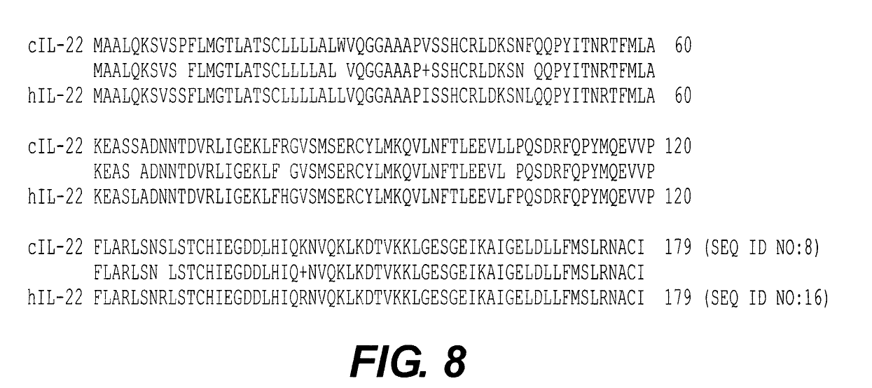

FIG. 9 shows a graph depicting the proliferation of rat CD3⁺ cells at varying concentrations of conditioned medium (CM) derived either from 293 cells expressing rat IL-21 (triangles) or from 293 cells that did not express rat IL-21 (circles). The X-axis depicts the % of conditioned medium (CM) in the total volume of cell culture medium used and the Y-axis depicts proliferation of cells as measured in counts per minute (CPMs) by incorporation of radioactive thymidine. As shown in the graph, the 50% maximum proliferation of rat CD3⁺ cells was observed at about 0.22% CM.

FIG. 10 shows a graph depicting the proliferation of rat CD3⁺ cells at varying concentrations of conditioned medium (CM) derived from 293 cells or COS-1 cells expressing rat IL-21 using either plasmid pED with the adenoviral late promoter or plasmid pSMED2 with the murine CMV promoter. As shown, rat CD3⁺ cells exhibit increased proliferation, as measured using incorporation of thymidine (Y-axis), in the presence of conditioned medium (CM) derived from either 293 or COS-1 cells (X-axis). Further, in the case of both cell-types, CM derived from cells expressing IL-21 using the plasmid pED appears to result in a higher proliferation than plasmid pSMED2 possibly because of increased expression.

FIG. 11 depicts a bar-graph which shows that at both concentrations 0.1% and 0.02%, CM derived from 293 cells expressing rat IL-21 (rat IL-21 CM) resulted in an increased secretion of cytokine IL-10 by rat CD3⁺ cells relative to the IL-10 secreted from cells that were treated with CM derived from 293 cells that did not express IL-21 (mock CM). The concentration of secreted IL-10 in the medium is measured in picograms/ml (pg/ml) using ELISA.

FIG. 12 depicts a graph which shows the proliferation (measured in CPMs on the Y-axis) of cynomolgus monkey peripheral blood leukocytes purified by ficoll-hypaque in the presence of varying concentrations of CM derived either from 293 cells or from COS-1 cells either expressing cynomolgus monkey IL-21 (293 cyno IL-21 CM and COS cyno IL-21 CM) or which do not express cynomolgus monkey IL-21 (mock CM). As shown, cynomolgus monkey cells treated with CM derived from either of 293 and COS-1 cells expressing cynomolgus monkey IL-21 show a proliferation about two to six fold higher than the proliferation of cells treated with mock CM. The two different cynomolgus IL-21 preparations are from different 293 batches.

FIG. 13 depicts a graph showing the secretion of GROa by human HT29 cells in the presence of titrating amounts of human IL-22 (huIL-22), murine IL-22 (muIL-22), rat IL-22 and cynomolgus IL-22 (monkey IL-22).

FIG. 14 depicts a graph showing the proliferation of BaF3 cells in the presence of increasing amounts of human IL-22 (huIL-22), murine IL-22 (muIL-22), rat IL-22 and cynomolgus IL-22 (monkey IL-22).

FIG. 15 shows an alignment between the nucleotide sequence of rabbit IL-22 (SEQ ID NO:39) and a human IL-22 sequence (SEQ ID NO:15), demonstrating about 85% identity between nt 1 to nt 559 of SEQ ID NO:39 and nt 72 to nt 633 of SEQ ID NO:15.

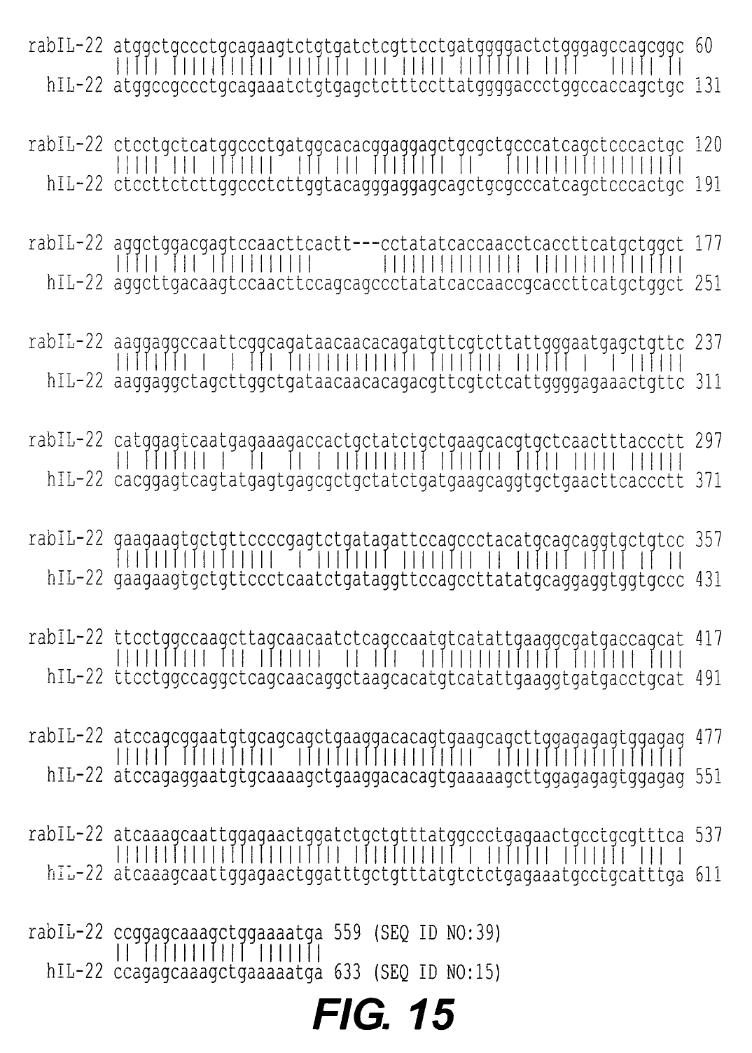

FIG. 16 shows an alignment between the amino acid sequence of rabbit IL-22 (SEQ ID NO:40) and a human IL-22 sequence (SEQ ID NO:16), demonstrating about 78% identity between aa 1 to aa 178 of SEQ ID NO:40 and aa 1 to aa 179 of SEQ ID NO:16.

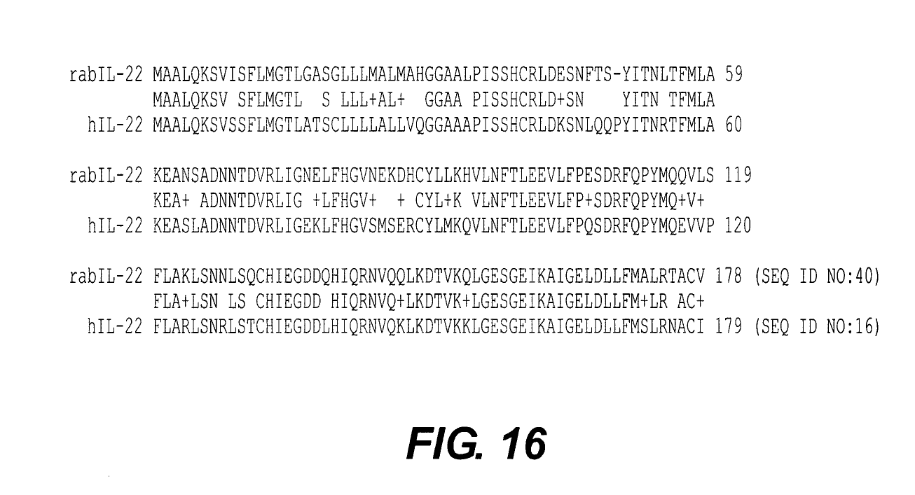

FIG. 17 depicts a graph showing the proliferation of BaF3 cells in the presence of increasing amounts of human IL-22 (huIL-22) and rabbit IL-22.

### DETAILED DESCRIPTION

In order that the present disclosure be more readily understood, certain terms are first defined. Additional definitions are set forth throughout the detailed description.

The term “isolated” as it applies to a nucleic acid molecule refers to a deoxyribonucleic acid, a ribonucleic acid, or a nucleic acid analog having a polynucleotide sequence that has been removed from its naturally occurring environment and is substantially free from other cellular material. An isolated nucleic acid encompasses nucleic acids that may be partially or wholly, chemically or recombinantly synthesized and/or purified by standard techniques known in the art. The term “isolated” as it applies to a protein refers to a polypeptide or a polypeptide analog having an amino acid sequence that has been removed from its naturally occurring environment and is substantially free from other cellular material.

The term “variant” in reference to a IL-21 or IL-22 nucleotide sequence refers to a nucleotide sequence that is substantially identical over the entire length to the nucleotide sequence set forth in SEQ ID NO:1, 3, 5, 7, 39, nucleotides 1 to 438 of SEQ ID NO:1, 52 to 438 of SEQ ID NO:1, 1 to 486 of SEQ ID NO:3, 88 to 486 of SEQ ID NO:3, 1 to 537 of SEQ ID NO:5, 100 to 537 of SEQ ID NO:5, 32 to 568 of SEQ ID NO:7, 131 to 568 of SEQ ID NO:7, 1 to 561 of SEQ ID NO:39, 100 to 561 of SEQ ID NO:39 or to its complementary strand over the entire length thereof, provided that the nucleotide sequence encodes a polypeptide with either IL-21 or IL-22 activity. The term “IL-21 activity” as it applies to a rat or cynomolgus IL-21 polypeptide refers to a biological activity of IL-21 as measured in a particular biological assay, with or without dose-dependency. Such an activity includes any known biological activity of IL-21, including but not limited to, for example, proliferation of CD3⁺ cells and secretion of IL-10 by CD3⁺ cells. An IL-21 activity encompasses a known activity of counterparts of rat and cynomolgus IL-21 polypeptides in other organisms such as, for example, mouse and human.

The term “IL-22 activity” as it applies to a rat, rabbit or cynomolgus IL-22 polypeptide refers to a biological activity of IL-22, as measured in a particular biological assay, with or without dose-dependency. Such an activity includes any known biological activity of IL-22 including but not limited to, for example, binding to an IL-22 receptor complex comprising IL-22R and IL-10R2, secretion of GROa by HT29 cells (as described in Example 9) and proliferation of IL-22 receptor engineered BaF3 cells (as described, for example, in Example 10). An IL-22 activity encompasses a known activity of counterparts of rat, rabbit and cynomolgus IL-22 polypeptides in other organisms such as, for example, mouse and human.

Variants of rat IL-21 nucleotide sequence may be the same length as the nucleotide sequence set forth in SEQ ID NO:1, nucleotides 1 to 438 or nucleotides 52 to 438 of SEQ ID NO:1, or shorter, so long as they encode a polypeptide with IL-21 activity. Variants of cynomolgus monkey IL-21 nucleotide sequence may be the same length as the nucleotide sequence set forth in SEQ ID NO:3, nucleotides 1 to 486 or nucleotides 88 to 486 of SEQ ID NO:3, or shorter, so long as they encode a polypeptide with IL-21 activity. Similarly, variants of rat IL-22 nucleotide sequence may be the same length as the nucleotide sequence set forth in SEQ ID NO:5, nucleotides 1 to 537 or nucleotides 100 to 537 of SEQ ID NO:5, or shorter, so long as they encode a polypeptide with IL-22 activity, variants of cynomolgus monkey IL-22 nucleotide sequence may be the same length as the nucleotide sequence set forth in SEQ ID NO:7, nucleotides 32 to 568 or nucleotides 131 to 568 of SEQ ID NO:7, or shorter, so long as they encode a polypeptide with IL-22 activity, and variants of rabbit IL-22 nucleotide sequence may be the same length as the nucleotide sequence set forth in SEQ ID NO:39, nucleotides 1 to 561 or nucleotides 100 to 561 of SEQ ID NO:39, or shorter, so long as they encode a polypeptide with IL-22 activity. Variants of the rat and cynomolgus monkey IL-21 nucleotide sequences can be naturally occurring, for example, naturally occurring sequences isolated from species other than mouse and human, or they can be generated artificially. Variants of the rat, rabbit and cynomolgus monkey IL-22 nucleotide sequences can also be naturally occurring, for example, naturally occurring sequences isolated from species other than mouse and human, or they can be generated artificially.

The identity between the rat IL-21 nucleotide sequence set forth in nucleotides 1 to 438 or nucleotides 52 to 438 of SEQ ID NO:1 and a variant thereof, when optimally aligned, is at least 95% identical (or 5% different), 96% identical (or 4% different), 97% identical (or 3% different), 98% identical (or 2% different), or 99% identical (or 1% different) over entire lengths of the variant and the sequence set forth in nucleotides 1 to 438 or nucleotides 52 to 438 of SEQ ID NO:1. Similarly, the identity between the cynomolgus monkey IL-21 nucleotide sequence set forth in nucleotides 1 to 486 or nucleotides 88 to 486 of SEQ ID NO:3 and a variant thereof is at least 95% identical (or 5% different), 96% identical (or 4% different), 97% identical (or 3% different), 98% identical (or 2% different) or 99% identical (or 1% different) over entire lengths of the variant and the sequence set forth in nucleotides 1 to 486 or nucleotides 88 to 486 of SEQ ID NO:3, provided that the variant does not comprise SEQ ID NO:11. The identity between the rat IL-22 nucleotide sequence set forth in nucleotides 1 to 537 or nucleotides 100 to 537 of SEQ ID NO:5 and a variant thereof, when optimally aligned, is at least 95% identical (or 5% different), 96% identical (or 4% different), 97% identical (or 3% different), 98% identical (or 2% different), or 99% identical (or 1% different) over entire lengths of the variant and the sequence set forth in nucleotides 1 to 537 or nucleotides 100 to 537 of SEQ ID NO:5; and identity between the cynomolgus monkey IL-22 nucleotide sequence set forth in nucleotides 32 to 568 or nucleotides 131 to 568 of SEQ ID NO:7 and a variant thereof is at least 95% identical (or 5% different), 96% identical (or 4% different), 97% identical (or 3% different), 98% identical (or 2% different), or 99% identical (or 1% different) over entire lengths of the variant and the sequence set forth in nucleotides 32 to 568 or nucleotides 131 to 568 of SEQ ID NO:7, provided that the variant does not comprise SEQ ID NO:15. The identity between the rabbit IL-22 nucleotide sequence set forth in nucleotides 1 to 561 or nucleotides 100 to 561 of SEQ ID NO:39 and a variant thereof, when optimally aligned, is at least 95% identical (or 5% different), 96% identical (or 4% different), 97% identical (or 3% different), 98% identical (or 2% different), or 99% identical (or 1% different) over entire lengths of the variant and the sequence set forth in nucleotides 1 to 561 or nucleotides 100 to 561 of SEQ ID NO:39.

The identity between the rat IL-21 amino acid sequence forth in SEQ ID NO:2 or amino acids 18 to 146 of SEQ ID NO:2 and a variant thereof, when optimally aligned, is at least 95% identical (or 5% different), 96% identical (or 4% different), 97% identical (or 3% different), 98% identical (or 2% different), or 99% identical (or 1% different) over entire lengths of the variant and the sequence set forth in SEQ ID NO:2 or amino acids 18 to 146 of SEQ ID NO:2. Similarly, the identity between the cynomolgus monkey IL-21 amino acid sequence set forth in SEQ ID NO:4 or amino acids 30 to 162 of SEQ ID NO:4 and a variant thereof is at least 97% identical (or 3% different), 98% identical (or 2% different) or 99% identical (or 1% different) over entire lengths of the variant and the sequence set forth in SEQ ID NO:4 or amino acids 30 to 162 of SEQ ID NO:4, provided that the variant does not comprise SEQ ID NO:12. The identity between the rat IL-22 amino acid sequence set forth in SEQ ID NO:6 or amino acids 34 to 179 of SEQ ID NO:6 and a variant thereof, when optimally aligned, is at least 95% identical (or 5% different), 96% identical (or 4% different), 97% identical (or 3% different), 98% identical (or 2% different), or 99% identical (or 1% different) over entire lengths of the variant and the sequence set forth in SEQ ID NO:6 or amino acids 34 to 179 of SEQ ID NO:6; and identity between the cynomolgus monkey IL-22 amino acid sequence set forth in SEQ ID NO:8 or amino acids 34 to 179 of SEQ ID NO:8 and a variant thereof is at least 95% identical (or 5% different), 96% identical (or 4% different), 97% identical (or 3% different), 98% identical (or 2% different), or 99% identical (or 1% different) over entire lengths of the variant and the sequence set forth in SEQ ID NO:8 or amino acids 34 to 179 of SEQ ID NO:8. The identity between the rabbit IL-22 amino acid sequence set forth in SEQ ID NO:40 or amino acids 34 to 187 of SEQ ID NO:40 and a variant thereof is at least 95% identical (or 5% different), 96% identical (or 4% different), 97% identical (or 3% different), 98% identical (or 2% different), or 99% identical (or 1% different) over entire lengths of the variant and the sequence set forth in SEQ ID NO:40 or amino acids 34 to 187 of SEQ ID NO:40. Variants of rat and cynomolgus monkey IL-21 nucleotide sequences may, for example, include homologs of these sequences in other species, including rodents and other mammals, but excluding mouse and human and known variants thereof. Variants of the rat IL-21 and IL-22 nucleotide sequences set forth in SEQ ID NOs:1 and 5 may also be found in other rodent species such as, for example, hamster, guinea pig, woodchuck, muskrat, gerbil, squirrel, chipmunk, prairie dog, beaver, porcupine, and vole, variants of the rabbit IL-22 nucleotide sequences set forth in SEQ ID NO:39 may also be found in other lagomorpha species such as, for example, hares and pikas and variants of cynomolgus monkey IL-21 and IL-22 nucleotide sequences set forth in SEQ ID NOs:3 and 7 may be found in other primate species, for example, baboon, chimpanzee, rhesus monkey, aotus monkey and african green monkey. Also contemplated are nucleotide sequences which hybridize to the IL-21 and IL-22 nucleotide sequences set forth in SEQ ID NOs:1, 3, 5, 7, and 39 under stringent hybridization conditions.

The term “transgenic” refers to any animal containing genetically manipulated cells in which an IL-21 DNA molecule and/or an IL-22 DNA molecule described herein is operably linked to a promoter which is not the same as the promoter to which the DNA molecule is linked in a naturally occurring genome. The term “transgenic” encompasses, for example, an animal containing cells which include an IL-21 and/or an IL-22 nucleotide sequence or a variant thereof described herein integrated within the animal's chromosomes. The term “transgenic” also encompasses an animal containing cells with an extrachromosomally replicating DNA sequence comprising an IL-21 or IL-22 nucleotide sequence described herein or a variant thereof. The transgenic animal may be a mammal such as a rodent or a primate.

Without wishing to be bound by theory, it is contemplated that such transgenic animals are expected to provide valuable insight into the potential pharmacological and toxicological effects in humans of compounds that are identified as agonists or antagonists of IL-21 or IL-22 activity. Additionally, an understanding of how IL-21 and IL-22 cytokines function in transgenic animal models is expected to provide an insight into treating and/or preventing human diseases that are associated with an increase or decrease in IL-21 or IL-22, including, but not limited to cancer, inflammatory and auto-immune diseases.

Also encompassed by this disclosure are “knock-out” animals, for example, a knock-out rat, knock-out rabbit or a knock-out cynomolgus monkey, which are lacking in one or more of a gene for IL-21 and/or IL-22. Such animals are useful as model systems for investigating biological activity of IL-21 and/or IL-22 and further for investigating disorders associated with a decrease in or absence of IL-21 and/or IL-22, and for screening for compounds useful for treating such disorders.

### Cloning of Sequences

The approach used for the isolation and cloning of the rat, rabbit and cynomolgus IL-21 and IL-22 nucleotide sequences described herein can be used for cloning homologs of these cytokines in other organisms and also for identifying variants of these sequences. For example, variants of the IL-21 and IL-22 nucleotide sequences described herein can be identified by hybridization to one or more of the sequences set forth in SEQ ID NOs:1, 3, 5, 7 or 39.

It is well known that the melting temperature (Tm) of a double stranded nucleic acid decreases by 1-1.5C with every 1% decrease in homology (see, e.g., Bonner et al. (1973) J. Mol. Biol., 81:123). Homologs in other species, therefore, can be identified, for example, by hybridizing a putative nucleotide sequence with a nucleotide set forth in SEQ ID NOs:1, 3, 5, 7 or 39, or a variant thereof, and comparing the melting temperature of such a hybrid with the melting temperature of a hybrid comprising a nucleotide sequence of SEQ ID NOs:1, 3, 5, 7 or 39 or a variant thereof and a complementary nucleotide sequence. The number of base pair mismatches can then be calculated for the test hybrid. Therefore, a smaller difference between the melting temperatures of the test hybrid and a hybrid containing a putative homolog of any one of sequences in SEQ ID NOs:1, 3, 5, 7 or 39 will indicate a greater homology between the putative nucleotide sequence and an IL-21 or IL-22 sequence of the invention.

For example, variants of rat IL-21 or IL-22 in other rodent species such as mouse, guinea pig, woodchuck, muskrat, gerbil, squirrel, chipmunk, prairie dog, beaver, porcupine, and vole, may exhibit a greater homology to the respective rat IL-21 or IL-22 sequences described herein. Variants of rabbit IL-22 in other lagomorpha species such as, for example, hares and pikas may exhibit a greater homology to the respective rabbit IL-22 sequences described herein. Similarly, variants of the cynomolgus monkey IL-21 or IL-22 in other primate species such as baboon, chimpanzee, rhesus monkey, aotus monkey and african green monkey, may exhibit a greater homology to the respective cynomolgus monkey IL-21 and IL-22 sequences described herein.

A variety of factors are known to affect the efficiency of hybridization of two strands of nucleotide sequence. These may include, for example, length of nucleotide sequence, salt concentration and G/C content of the sequences. For example, for hybridization of long fragments of DNA, Howley et al. (1979) J. Biol. Chem., 254:4876, determined that the melting temperature at which 50% of a DNA is hybridized to a complementary strand is defined by:

Tₘ=81.5+16.6 log M+41(% G+% C)−500/L−0.62F, where

M is molar concentration of monovalent cations;

(% G+% C) is the respective fraction of G and C nucleotides in the sequences;

L is length of the hybrid DNA; and

F is molar concentration of formamide.

Appropriate hybridization conditions can be selected by those skilled in the art with minimal experimentation as exemplified in Ausubel et al. (1995) Current Protocols in Molecular Biology, John Wiley & Sons, sections 2, 4, and 6. Additionally, stringent conditions are described in Sambrook et al. (1989) Molecular Cloning: A Laboratory Manual, 2nd ed., Cold Spring Harbor Press, chapters 7, 9, and 11.

A non-limiting example of low stringency hybridization conditions is as follows. Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll™, 1% BSA, and 500 μg/ml denatured salmon sperm DNA. Hybridizations are carried out in the same solution with the following modifications: 0.02% PVP, 0.02% Ficoll™, 0.2% BSA, 100 μg/ml salmon sperm DNA, 10% (wt/vol) dextran sulfate, and 5-20×106 32P-labeled probe is used. Filters are incubated in hybridization mixture for 18-20 h at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated for an additional 1.5 hours at 60° C. Filters are blotted dry and exposed for autoradiography. Other conditions of low stringency well known in the art may be used (e.g., as employed for cross species hybridizations).

A non-limiting example of high stringency hybridization conditions is as follows. Prehybridization of filters containing DNA is carried out for 8 h to overnight at 65° C. in buffer containing 6×SSC, 50 mM Tris-HCl (pH 7.5), 1 mM EDTA, 0.02% PVP, 0.02% Ficoll™, 0.02% BSA, and 500 μg/ml denatured salmon sperm DNA. Filters are hybridized for 48 hours at 65° C. in the prehybridization mixture containing 100 μg/ml denatured salmon sperm DNA and 5-20×10⁶ cpm of ³²P-labeled probe. Washing of filters is done at 37° C. for 1 hours in a solution containing 2×SSC, 0.01% PVP, 0.01% Ficol™, and 0.01% BSA. This is followed by a wash in 0.1×SSC at 50° C. for 45 minutes.

A non-limiting example of hybridization conditions of moderate stringency includes prewashing filters in 5×SSC, 0.5% SDS, 1.0 mM EDTA, pH 8.0; hybridizing in 50% formamide, 6×SSC at 42° C.; and washing filters in 0.5×SSC, 0.1% SDS at 60° C.

Variants of the IL-21 and IL-22 DNA molecules described herein can also be identified by percent identity between nucleotide sequences for putative variants and the sequences set forth in SEQ ID NOs:1, 3, 5, 7, 39, nucleotides 1 to 438 of SEQ ID NO:1, 52 to 438 of SEQ ID NO:1, 1 to 486 of SEQ ID NO:3, 88 to 486 of SEQ ID NO:3, 1 to 537 of SEQ ID NO:5, 100 to 537 of SEQ ID NO:5, 32 to 568 of SEQ ID NO:7, 131 to 568 of SEQ ID NO:7, 1 to 561 of SEQ ID NO:39, 100 to 561 of SEQ ID NO:39 or their complementary strands. Percent identity may be determined, for example, by visual inspection or by using various computer programs known in the art or as described herein. For example, percent identity of two nucleotide sequences can be determined by comparing sequence information using the GAP computer program described by Devereux et al. (1984) Nucl. Acids. Res., 12:387 and available from the University of Wisconsin Genetics Computer Group (UWGCG). Percent identity can also be determined by aligning two nucleotide sequences using the BLAST® program as described by Tatusova et al. (1999) FEMS Microbiol. Lett., 174:247 and available at the National Center for Biotechnology website. For example, for nucleotide sequence alignments using the BLAST® program, the default settings are as follows: reward for match is 1, penalty for mismatch is −3, open gap and extension gap penalties are 5 and 2 respectively, gap×dropoff is 50, expect is 10, word size is 11, and filter is OFF.

IL-21 nucleotide sequences identified as being most identical to the sequence set forth in SEQ ID NO:1 (rat IL-21) or SEQ ID NO:3 (monkey IL-21) include for example, nucleotide 1 to 441 of mouse IL-21 sequence set forth in SEQ ID NO:9, that shows about 91% identity to nucleotide 1 to 441 of rat IL-21 sequence set forth in SEQ ID NO:1, and nucleotide 46 to 534 of human IL-21 sequence set forth in SEQ ID NO:11, that shows about 98% identity to nucleotide 1 to 489 of SEQ ID NO:3. Similarly, IL-22 nucleotide sequences identified as being most identical to the sequence set forth in SEQ ID NO:5 (rat IL-22), or SEQ ID NO:7 (monkey IL-22) include for example, nucleotide 54 to 590 of mouse IL-22 sequence set forth in SEQ ID NO:13, that shows about 90% identity to nucleotide 1 to 537 of SEQ ID NO:5, and nucleotide 72 to 633 of human IL-22 sequence set forth in SEQ ID NO:15, that shows about 96% identity to nucleotide 1 to 653 of SEQ ID NO:5. IL-22 nucleotide sequences identified as being most identical to the sequence set forth in SEQ ID NO:39 (rabbit IL-22) include for example, nucleotide 72 to 633 of human IL-22 sequence set forth in SEQ ID NO:15, that shows about 85% identity to nucleotide 1 to 559 of SEQ ID NO:39. Also, additional sequences may be readily identified using the various techniques described herein and those known in the art.

Percent identity between the nucleotide sequences set forth in this application, such as SEQ ID NOs:1, 3, 5, 7, or 39 and similar sequences can be determined as described herein. For example, when the nucleotide sequence set forth in SEQ ID NO:1 is compared to other known nucleotide sequences using BLAST® sequence alignment with default parameters, notable examples include the mouse IL-21 sequence set forth in Genbank® Accession No. AY428162.1, which exhibits about 91% identity to SEQ ID NO:1 over the entire length, and Genbank® Accession No. XM_{—}345201.1, which exhibits about 40% identity to SEQ ID NO:1 over the entire length of the sequence. When the sequence set forth in SEQ ID NO:3 is compared to other known nucleotide sequences using BLAST® sequence alignment with default parameters, it exhibits about 87% identity over the entire length of human IL-21 sequence set forth in Genbank® Accession No. BC066261.1. Similarly, sequences set forth in SEQ ID NO:5 and 7 exhibit 90% and 96% identity, respectively, over the entire length of sequences in Genbank® Accession Nos. NM_{—}016971 and BC069112, respectively. Similarly, the sequence set forth in SEQ ID NO:39 exhibits 85% identity over the entire length of sequences in Genbank® Accession No. BC069112.

Percent identity between the amino acid sequences set forth in this application, such as SEQ ID NOs:2, 4, 6, 8 and 40 and known amino acid sequences can also be determined using BLAST® sequence alignment with default parameters. When the rat IL-21 amino acid sequence set forth in SEQ ID NO:2 is compared to other known amino acid sequences using BLAST® sequence alignment with default parameters, it exhibits about 87% identity over the entire length of the mouse IL-21 sequence in Genbank® Accession No. MR06254.1. When the cynomolgus amino acid sequence set forth in SEQ ID NO:4 is compared to other known amino acid sequences using BLAST® sequence alignment with default parameters, it exhibits about 96% identity over the entire length of the human IL-21 amino acid sequence in Genbank® Accession No. MU88182.1. When the rat IL-22 amino acid sequence set forth in SEQ ID NO:6 is compared to other known amino acid sequences using BLAST® sequence alignment with default parameters, aa 1 to aa 179 of the sequence set forth in SEQ ID NO:6 exhibits about 90% identity over the entire length of the mouse IL-22 sequence in Genbank® Accession No. NP_{—}058667.1. Similarly, when the cynomolgus IL-22 amino acid sequence set forth in SEQ ID NO:8 is compared to other known amino acid sequences using BLAST® sequence alignment with default parameters, it exhibits about 94% identity over the entire length of human IL-22 sequence in Genbank® Accession No. BC066265. Similarly, when the rabbit IL-22 amino acid sequence set forth in SEQ ID NO:40 is compared to other known amino acid sequences using BLAST® sequence alignment with default parameters, it exhibits about 78% identity over the entire length of human IL-22 sequence in Genbank® Accession No. BC066265.

Accordingly, in some embodiments, an IL-21 nucleotide sequence or a variant thereof comprises from nucleotide 1 to 438 of SEQ ID NO:1, from nucleotide 52 to 438 of SEQ ID NO:1, from nucleotide 1 to 486 of SEQ ID NO:3, or from nucleotide 88 to 486 of SEQ ID NO:3. In some embodiments, an IL-22 sequence or a variant thereof comprises from nucleotide 1 to 537 of SEQ ID NO:5, from nucleotide 100 to 537 of SEQ ID NO:5, from nucleotide 32 to 568 of SEQ ID NO:7, from nucleotide 131 to 568 of SEQ ID NO:7, or from nucleotide 1 to 561 of SEQ ID NO:39 or from nucleotide 100 to 561 of SEQ ID NO:39.

In some embodiments, an IL-21 polypeptide or a variant thereof comprises an amino acid sequence from amino acid 1 to 146 of SEQ ID NO:2, from amino acid 18 to 146 of SEQ ID NO:2, from amino acid 1 to 162 of SEQ ID NO:4, or from amino acid 30 to 162 of SEQ ID NO:4. Similarly, in some embodiments, an IL-22 polypeptide or a variant thereof comprises an amino acid sequence from amino acid 1 to 179 of SEQ ID NO:6, from amino acid 34 to 179 of SEQ ID NO:6, from amino acid 1 to 179 of SEQ ID NO:8, from amino acid 34 to 179 of SEQ ID NO:8, from amino acid 1 to 187 of SEQ ID NO:40, or from amino acid 34 to 187 of SEQ ID NO:40.

Nucleotide sequences set forth in SEQ ID NOs:1, 3, 5, 7 or 39, or variants thereof, can be used as probes for screening cDNA expression libraries for the isolation of sequences that hybridize to one or more of these sequences.

The various nucleotide and amino acid sequences discussed herein are listed in Table I below.

TABLE I
Sequences

| Sequence Name | SEQ ID NO. |
|---|---|
| Rat IL-21 cDNA | SEQ ID NO: 1 |
| Rat IL-21 protein | SEQ ID NO: 2 |
| Monkey IL-21 cDNA | SEQ ID NO: 3 |
| Monkey IL-21 protein | SEQ ID NO: 4 |
| Rat IL-22 cDNA | SEQ ID NO: 5 |
| Rat IL-22 protein | SEQ ID NO: 6 |
| Monkey IL-22 cDNA | SEQ ID NO: 7 |
| Monkey IL-22 protein | SEQ ID NO: 8 |
| Murine IL-21 cDNA (Accession No. AY428162.1) | SEQ ID NO: 9 |
| Murine IL-21 protein (Accession No. AAR06254.1) | SEQ ID NO: 10 |
| Human IL-21 cDNA (Accession No. BC066261.1) | SEQ ID NO: 11 |
| Human IL-21 protein (Accession No. AAU88182.1) | SEQ ID NO: 12 |
| Murine IL-22 cDNA (Accession No. NM_016971) | SEQ ID NO: 13 |
| Murine IL-22 protein (Accession No. NP_058667.1) | SEQ ID NO: 14 |
| Human IL-22 cDNA (Accession No. BC069112) | SEQ ID NO: 15 |
| Human IL-22 protein (Accession No. BC066265) | SEQ ID NO: 16 |
| Rabbit IL-22 cDNA | SEQ ID NO: 39 |
| Rabbit IL-22 protein | SEQ ID NO: 40 |

Modifications can be made in the polypeptides described herein by making conservative amino acid modifications which result in polypeptides having functional and chemical characteristics similar to those of the molecule from which such modifications are made. In contrast, substantial modifications in the functional and/or chemical characteristics of the polypeptides may be accomplished by selecting substitutions in the amino acid sequence that differ significantly in their effect on maintaining the structure of the polypeptide, the charge or hydrophobicity of the polypeptide and/or the size of the polypeptide.

For example, a “conservative amino acid substitution” may involve a substitution of a native amino acid residue with a normative residue such that there is little or no effect on the polarity or charge of the amino acid residue at that position. In certain embodiments, conservative amino acid substitutions also encompass non-naturally occurring amino acid residues which are typically incorporated by chemical peptide synthesis rather than by synthesis in biological systems.

### Composition and Modification of Sequences

IL-21 and IL-22 nucleotide molecules described herein can be composed of either polyribonucleotide or polydeoxyribonucleotide, which may comprise modified or unmodified bases. Examples of modified bases, include, for example, tritylated bases and unusual bases such as inosine. Nucleotide sequences described herein may either be chemically synthesized or they can be derived from a natural source.

IL-21 and IL-22 polypeptides described herein can be composed of amino acids joined to each other by peptide bonds or by modified peptide bonds, and may contain amino acids other than the 20 naturally occurring amino acids. The IL-21 and IL-22 polypeptides may be modified by naturally occurring processes, such as, posttranslational processing or may be modified synthetically using chemical modification techniques that are known in the art. Such modifications may occur anywhere in the polypeptides, including the peptide backbone, the amino acid side-chains and/or the amino or carboxyl termini. Examples of modifications include, but are not limited to, acetylation, acylation, ADP-ribosylation, amidation, covalent attachment of oligosaccharide or lipids, glycosylation, ubiquitination, pegylation, proteolytic processing, methylation, demethylation, phosphorylation, prenylation, racemization, sulfation and disulfide-bond formation. Polypeptides described herein may either be chemically synthesized or be derived from a natural source.

IL-21 and IL-22 polypeptides described herein and variants thereof can be fused to other proteins, for example, to generate a fusion protein. These fusion proteins can be used for a variety of applications. For example, fusions of polypeptides to an IgG molecule, such as, for example, IgG1 or IgG3, can be used for increasing half-life of the polypeptides in vivo. Fusions of IL-21 to IL-22 to tags including but not limited to, Flag-tag, His-tag, HA-tag, protein A and maltose binding protein (MBP), can be used to facilitate purification of the polypeptides. Additionally, fusions can be generated which increase solubility and/or stability of the polypeptides or which target the polypeptides to a particular subcellular location in a cell.

### Uses for the Sequences of the Invention

IL-21 and IL-22 polynucleotides described herein can also be used as molecular weight markers on Southern gels or as diagnostic probes to detect the presence of an IL-21 or IL-22 nucleic acid in a biological sample. Additionally, such polynucleotides can be used in experiments involving subtractive hybridization to select novel polynucleotides, for example, or for raising anti-IL-21 DNA or anti-IL-22 DNA antibodies by using such polynucleotides as an antigen.

Polypeptides described herein can be used for generating antibodies which selectively bind the polypeptide. Antibodies that selectively bind polypeptides described herein can either be polyclonal antibodies or monoclonal antibodies. Antibodies encompassed by this disclosure also include antigen-binding antibody fragments such as, Fab, Fab′, F(ab′)2, scFv, disulfide-linked Fvs and fragments comprising either a V_{L} or a V_{H} domain. Antibodies may be chimeric and/or humanized.

Antibodies that specifically bind to an IL-21 or IL-22 polypeptide comprising an amino acid sequence set forth in SEQ ID NO:2, 4, 6, 8, 40, amino acids 18 to 146 of SEQ ID NO:2, amino acids 30 to 162 of SEQ ID NO:4, amino acids 34 to 179 of SEQ ID NO:6, amino acids 34 to 179 of SEQ ID NO:8, or amino acids 34 to 187 of SEQ ID NO:40; however, which do not specifically bind to any other analog, ortholog or homolog of these polypeptides are encompassed by this disclosure. For example, an IL-21 antibody encompasses antibodies that selectively bind a polypeptide comprising the amino acid sequence set forth in SEQ ID NO:2 or 4, but not to a polypeptide which comprises an amino acid sequence which is less than 95% identical (or greater than 5% different) to SEQ ID NO:2 or an amino acid sequence which is less than 97% identical (or greater than 3% different) to SEQ ID NO:4. Similarly, an IL-22 antibody encompasses antibodies that selectively bind a polypeptide comprising the amino acid sequence set forth in SEQ ID NO:6, 8, or 40 but not to a polypeptide which comprises an amino acid sequence which is less than 95% identical (or greater than 5% different) than SEQ ID NO:6, 8 or 40.

IL-21 and IL-22 nucleotides and polypeptides described herein can be used for identifying compounds including, for example, agonists and antagonists/inhibitors of these cytokines. Such compounds can be used for generating in vitro and/or in vivo data useful for predicting pharmacokinetics of such compounds in humans for agonizing or antagonizing biological activity of IL-21 or IL-22.

In some embodiments, IL-21 polypeptides described herein can be administered to a subject having a disorder associated with a decrease in an IL-21 activity relative to such activity in a subject not having such a disorder. Similarly, IL-22 polypeptides described herein can be administered to a subject having a disorder associated with a decrease in an IL-22 activity relative to such activity in a subject not having such a disorder. Compositions and methods further relate to the use of IL-21 and IL-22 in various therapeutic applications, including use of IL-22 polypeptides and nucleic acids in the treatment of cancer (such as leukemia, lymphoma, and solid tumors), infections (such as bacterial, viral, or parasitic infection), or conditions associated with a suppressed immune response (due to infection, such as HIV infection; chemotherapy, bone marrow transplant, organ transplant, hyposplenism, malnutrition and chronic disease, nephritic syndrome, and premature birth, severe and other combined immunodeficiences, di George syndrome, Wiskott Aldrich Syndrome, ataxia telangiectasia, leukocyte adhesion deficiency, hyper IgM syndrome, chronic mucocutaneous candidiasis, hyper IgE syndrome, familial ertythrophagocytic lymphohistiocytosis, X linked agammaglobulinaemia, common variable immunodeficiency, IgA deficiency, IgG deficiency, chronic neutropenia, chronic granulomatous disease, complement deficiency disease, opsonization defects). IL-21 and/or IL-22 polypeptides described herein can be administered to a subject in an effort to replace absent or decreased levels of one or both of these cytokines.

Antibodies to these cytokines may be used to treat autoimmune conditions, such as autoimmune disease disorders, inflammatory disorders, allergies, transplant rejection, cancer, immune deficiency, and other disorders. For example, antibodies to these cytokines can be used to treat a subject with an immune cell-associated disorder such as an autoimmune disorder (e.g., multiple sclerosis, diabetes mellitus (type I), arthritic disorders such as rheumatoid arthritis, juvenile rheumatoid arthritis, osteoarthritis, psoriatic arthritis, and ankylosing spondylitis); respiratory disorder (e.g., asthma, chronic obstructive pulmonary disease (COPD)); inflammatory conditions of, e.g., the skin (e.g., psoriasis), cardiovascular system (e.g., atherosclerosis), nervous system (e.g., Alzheimer's disease), kidneys (e.g., nephritis), liver (e.g., hepatitis) and pancreas (e.g., pancreatitis).

The cytokines and the antibodies can also be used to treat animals, such as mammals, farm animals, sporting animals, family pets, zoo animals, etc.

A subject having a disorder associated with a decrease or an increase in an IL-21 and/or IL-22 activity can be identified, for example, by assaying IL-21 and/or IL-22 activity or expression level in cells or a body fluid of the subject and comparing such activity or expression level with a standard activity or expression level for IL-21 and/or IL-22, where an increase or decrease in such activity or expression level is indicative of the disorder. A standard activity or expression level can be determined, for example, by measuring such activity or level in cells or a body fluid from an individual not having such a disorder.

In other embodiments, IL-21 and IL-22 polypeptides described herein can be used for identifying compounds which modulate, for example, decreased IL-21 or IL-22 activity. Such compounds can then be administered to a subject having a disorder associated with an increased IL-21 or IL-22 activity relative to such activity in a subject not having such a disorder.

### Nucleic Acid Construct and Expression

Nucleic acid sequences encoding the IL-21 and IL-22 sequences are provided in this invention as described above. Suitable vectors may be chosen or constructed to contain appropriate regulatory sequences, including promoter sequences, terminator sequences, polyadenylation sequences, enhancer sequences, marker genes, and other sequences. The vectors may also contain a plasmid or viral backbone. For details, see Sambrook et al., Molecular Cloning: A Laboratory Manual, 2nd ed., Cold Spring Harbor Laboratory Press (1989). Many established techniques used with vectors, including the manipulation, preparation, mutagenesis, sequencing, and transfection of DNA, are described in Current Protocols in Molecular Biology, Second Edition, Ausubel et al. eds., John Wiley & Sons (1992).

A further aspect of the disclosure provides a method of introducing the nucleic acid into a host cell. For eukaryotic cells, suitable transfection techniques may include calcium phosphate, DEAE-Dextran, electroporation, liposome-mediated transfection, and transduction using retrovirus or other viruses, e.g., vaccinia or baculovirus. For bacterial cells, suitable techniques may include calcium chloride transformation, electroporation, and transfection using bacteriophage. DNA introduction may be followed by a selection method (e.g., drug resistance) to select cells that contain the nucleic acid.

The following examples provide illustrative embodiments of the compositions and methods described herein. One of ordinary skill in the art will recognize the numerous modifications and variations that may be performed without altering the spirit and scope of the present invention. Such modifications and variations are encompassed within the scope of the invention. The examples are not in any way limiting.

### EXAMPLES

### Example 1

### Cloning of Rat IL-21 cDNA

cDNA encoding rat IL-21 was amplified using polymerase chain reaction (PCR) and further re-amplified from a pool of cDNAs derived from rat thymus, lung and testes. (Seegene, Inc., Seoul, South Korea).

The following primers were used for amplification with the KOD polymerase according to the manufacturer's instructions. (Novagen, Madison, Wis.).

5′-GTACAAAAAAGCAGGCTCCACCATGGAGAGGACCCTTGTCTGTC-3′ (SEQ ID NO:17); and

5′-ACTTTGTACAGAAAGCTGGGTCTAGGAAAGATGCTGATGATC-3′ (SEQ ID NO:18). The primer shown in SEQ ID NO:17 included an embedded partial Gateway® attB1 site and the primer shown in SEQ ID NO:18 included an embedded partial Gateway® attB2 site.

The PCR product (485 base pairs) was reamplified with primers 5′-GGGGACAGTTTGTACAAAAAAGCAGGCTCCACCATG-3′ (SEQ ID NO:37) and 5′-GGGGACCACTTTGTACAAGAAAGCTGGGT-3′ (SEQ ID NO:38) and was cloned into the Gateway® recombination cloning platform (Invitrogen, Carlsbad, Calif.) using BP CLONASE™ (Invitrogen), first into the Gateway® entry plasmid, pDONR221, and ultimately into plasmids pED and pSMED2 using LR CLONASE™ (Invitrogen). Multiple independently derived PCR products were sequenced to arrive at a consensus sequence shown in SEQ ID NO:1.

### Example 2

### Cloning of Cynomolgus Monkey IL-21 cDNA

Cynomolgus monkey IL-21 cDNA was amplified by PCR using a 1^{st} strand cDNA synthesis kit (Invitrogen, Carlsbad, Calif.). Total RNA was purified using RNA purification kit (Qiagen, Valencia, Calif.) from cynomoigus spleen cells activated with Conconavalin A in culture for 24 hours. The 5′ and 3′ untranslated regions of the corresponding human IL-21 gene sequence in Genbank® Accession No. NM_{—}021803 were used to design the 5′ and 3′ primers for the initial amplification. Primers 5′-GCTGAGTGAAAACGAGACCAAGG-3′ (SEQ ID NO:19) and 5′-GATACAAAGAAATGACTTTCACTAC-3′ (SEQ ID NO:20) were used to perform the first round of PCR using iProof™ (formerly Phusion) polymerase (Bio-Rad, Hercules, Calif.). Nested PCR was used to obtain sufficient amount for cloning. The following primers were used for re-amplification of the first PCR product: 5′-AAACGAGACCAAGGTCTAGCTCTAC-3′ (SEQ ID NO:21); and 5′-ATTAGAGTATGTACATAGTGTCC-3′ (SEQ ID NO:22). Purified PCR product of about 566 bp was subsequently cloned into plasmid pCRBluntII-top( ) (Invitrogen). Multiple independently derived PCR products were sequenced to arrive at a consensus sequence, shown in SEQ ID NO:3.

The cDNA encoding cynomolgus monkey IL-21 coding region was cloned into the Gateway® cloning platform by PCR using Gateway® attB sites embedded primers: 5′-GGGGACAGTTTGTACAAAAAAGCAGGCTATATGA GATCCAGTCCTGGCAACATG-3′ (SEQ ID NO:23); and 5′-GGGGACCACTTTGTAC

AGAAAGCTGGGTATCACTAGGGATCTTCACTTCCGTGTGTTCT-3′ (SEQ ID NO:24). The cDNA was subsequently cloned first into plasmid pDONR221 using BP CLONASE™ and ultimately into the pSMED2 vector using LR CLONASE™. Multiple independently derived PCR products were sequenced to arrive at a consensus sequence shown in SEQ ID NO:3.

### Example 3

### Cloning of Rat-IL-22 cDNA

The cDNA encoding rat IL-22 was PCR amplified and further re-amplified from a cDNA pool derived from rat thymus, lung and testis. (Seegene, Inc., Seoul, South Korea).

PCR primers 5′-CACCATGTCTGTCCTGAGGAAATCTATGAGC-3′ (SEQ ID NO:25) and 5′-GTGGTGGTGGTGATGGTGGGACCCCGACCCTGCGA CGCAAGCGTTTCTCAGGG-3′ (SEQ ID NO:26), which includes a 2×(glycine-serine) linker (SEQ ID NO: 45) and a poly His6x tag (SEQ ID NO: 46), were used do the first round of amplification using Advantage2 polymerase (Clontech, Palo Alto, Calif.) according to the manufacturer's protocols. The PCR product (568 bp) was gel purified and re-amplified using primers 5′-GGGGACAGTTTGTACAAAAAAGCAGGCTCCACCATGTCTGTCCTG AGGAAATC-3′ (SEQ ID NO:27) and 5′-GGGGACCACTTTGTACAGAAAGCTGGG TTCACTTGTCGTCATCGTCTTTGTAGTCGTGGTGGTGGTGATGGTG-3′ (SEQ ID NO:28) including Gatewaye attB1 plus Kozak consensus sequence and attB2 plus FLAG tag sequence, respectively. The resultant PCR product (654 bp) was cloned using the Gateway® recombination cloning platform (Invitrogen, Carlsbad, Calif.) into plasmid pDONR221 using BP CLONASE™. Multiple independently derived PCR products were sequenced to arrive at a consensus sequence shown in SEQ ID NO:5.

### Example 4

### Cloning of Cynomolgus Monkey IL-22 cDNA

cDNA encoding the cynomolgus monkey IL-22 was isolated using PCR amplification from a cDNA pool synthesized from total RNA isolated from cynomolgus monkey spleen cells that were activated with Conconavalin A for 24 hours in culture, using a 1^{st} strand cDNA synthesis kit (Invitrogen). Total RNA was isolated using total RNA purification kit (Qiagen, Valencia, Calif.).

Primers used for amplification, 5′-ACCAGGTTCTCCTTCCCCAG-3′ (SEQ ID NO:29) and 5′-GGCTTCCCATCTTCCTTTTGG-3′ (SEQ ID NO:30) were derived from the 5′ and 3′ untranslated regions (UTRs) of the human IL-22 gene with Genbank® Accession No. NM_{—}020525 and were used in a PCR reaction with the KOD polymerase (Novagen, Madison, Wis.). The purified PCR product of 697 bp was cloned into the pCRBluntII-topo plasmid (Invitrogen). Multiple independently derived PCR products were sequenced to arrive at the consensus sequence shown in SEQ ID NO:7.

The cynomolgus IL-22 cDNA was subsequently amplified by PCR using nested primers including the signal peptide of honeybee prepromelittin and His6x-FLAG tags (SEQ ID NO: 46) and Gateway® attB sites using the following primers: 5′-GGGGACAGTTTGTACAAAAAAGCAGGCTCCACCATGAAATTCTTAGTCA CGTTGCCCTTGTTTTT ATGGTCGTGTACATTTCTTACATCTATGCG-3′ (SEQ ID NO:31), which contains the Gateway® attB1 site and the honeybee prepromelittin signal peptide sequence; 5′-ATTT CTTACATCTATGCGGGTAGCGGCCACCATCACCA CCACCAC-3′ (SEQ ID NO:32), which contains a 2×(glycine-serine) linker (SEQ ID NO: 45), a His6x tag (SEQ ID NO: 46), and a partial overlapping sequence to the signal sequence; 5′-CACCATCACCACCA CCACGGTAGCGGCGACTACAAAGACGATGAC-3′ (SEQ ID NO:33), which contains a His6x tag (SEQ ID NO: 46), another 2×(glycine-serine) linker (SEQ ID NO: 45), and a partial overlapping FLAG tag sequence; 5′-GACTACAAAGACGATGACGACAGGCGCCCGTCAGCTCCCACTG-3′ (SEQ ID NO:34), which contains a FLAG tag sequence and a cynomolgus monkey IL-22 specific sequence; 5′-CTTTGTACAGAAAGCTGGGTTCAAATGCAGG

CATTTCTCAGAG-3′ (SEQ ID NO:35), which contains a cynomolgus monkey IL-22 specific sequence, a stop codon, and a partial overlapping Gateway® attB2 site; and 5′-GGGGACCACTTTGTACAGAAAGCTGGGT-3′ (SEQ ID NO:36), which contains a full Gateway® attB2 site, and cloned into the Gateway® cloning platform into the pDONR221 plasmid using BP CLONASE™ and ultimately into the pSMED2 plasmid using LR CLONASE™. Multiple independently derived PCR products were sequenced to arrive at a consensus sequence shown in SEQ ID NO:7.

### Example 5

### Preparation of Conditioned Medium from COS-1 Cells and 293 Cells Expressing Either Rat IL-21, Cynomolgus Monkey IL-21, or Rabbit IL-22

A 100 mm tissue culture plate of confluent COS-1 or 293 cells was split at a ratio of 1:4 for COS-1 cells and 1:3 for 293 cells in a final volume of about 8 mls. The cells were grown until about 80-90% confluent. COS-1 cells and 293 cells were transfected with plasmid containing DNA encoding either rat IL-21, cynomolgus monkey IL-21, or rabbit IL-22 using the TransIT® reagent (Mirus Bio Corporation, Madison, Wis.). About 40 μl of the TransiT® reagent was diluted into 2 mls GIBCO® OptiMEM® (Invitrogen) supplemented with 2 mM glutamine. The mixture was vortexed and incubated at room temperature for about 15 minutes. DNA encoding either rat IL-21 or cynomolgus monkey IL-21 was subsequently added to the mixture at a concentration of 16 μg DNA/2 mls for each plate. The TransIT®/OptiMEM®/DNA mixture was incubated at room temperature for about 15 minutes and was subsequently added to each plate of cells. Cells were incubated at 37° C., 10% CO₂ for approximately 20-24 hours. Medium was removed from each plate, cells were rinsed with about 10 mls GIBCO® RICD1 medium (Invitrogen) and about 10 mls of fresh RICD1 serum free medium supplemented with 100 μg/ml penicillin and 100 μg/ml streptomycin and 2 mM glutamine was added to the cells. Conditioned medium was harvested from COS-1 and 293 cells expressing either rat IL-21, cynomolgus monkey IL-21, or rabbit IL-22 after about 48 hours. The medium was centrifuged at 1200 rpm for 10 minutes to remove any cells from the conditioned medium.

### Example 6

### Rat IL-21 Stimulates Proliferation of Rat CD3⁺ Cells

Rat CD3⁺ cells were isolated from a male Sprague-Dawley rat spleen using a rat CD3⁺ enrichment column (R & D Systems, Inc., Minneapolis, Minn.). 1×10⁵ rat CD3⁺ cells suspended in Dulbecco's Modified Eagle's (DME) medium containing 10% fetal calf serum (JRH Biosciences, Inc., Lenexa, Kans.) were plated onto a 96 well plate with each well pre-coated with anti-rat CD3⁺ antibody (BD Pharmingen, San Diego, Calif.) and treated either with 0.1% conditioned medium from 293 cells expressing rat IL-21 or 0.1% conditioned medium from 293 cells that do not express rat IL-21. Cells were grown for three days. During the last five hours before harvesting, cells were labeled with 0.5 μCi methyl-3-thymidine/well (Amersham, Piscataway, N.J.). The cells were subsequently harvested using a Tomtech Mach III plate harvester (Wallac, Gaithersburg, Md.) and counted using a Perkin Elmer 1450 microbeta counter (Perkin Elmer, Wellesley, Mass.).

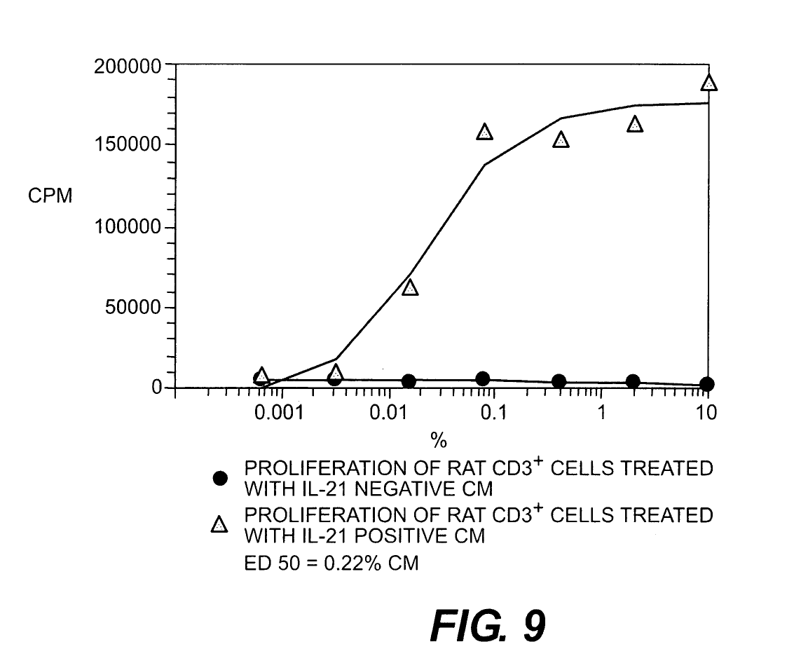

As shown in FIG. 9, rat CD3⁺ cells showed 50% of maximum proliferation at a concentration of about 0.22% conditioned medium from 293 cells expressing IL-21. In contrast, rat CD3⁺ cells grown in the presence of conditioned medium from 293 cells which did not express IL-21 showed relatively little to no proliferation even at a concentration as high as 10%.

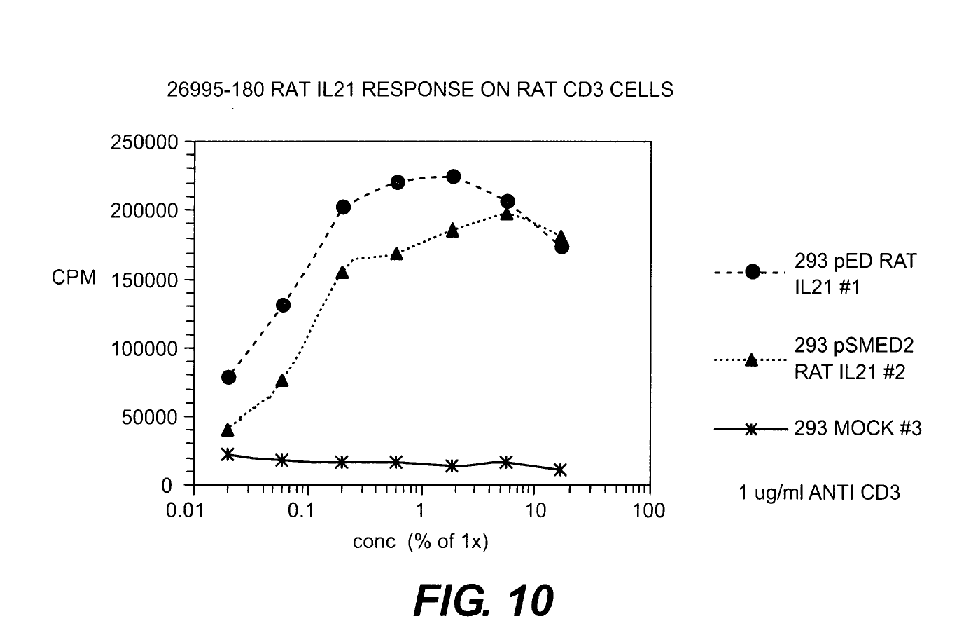

In order to further ensure that the effect on proliferation of the rat CD3⁺ cells was a property of the conditioned medium from cells expressing IL-21 and not that of the cell-type, in yet another experiment, rat CD3⁺ cells were treated with conditioned medium either from COS-1 cells expressing IL-21 or from 293 cells expressing IL-21. As depicted in FIG. 10, both conditioned media resulted in the proliferation of rat CD3⁺ cells when compared to cells grown in the presence of the conditioned medium which came from cells that did not express IL-21.

### Example 7

### Rat IL-21 Induces Rat CD3⁺ Cells to Secrete IL-10

Rat CD3⁺ cells were isolated from a male Sprague-Dawley rat spleen using a rat CD3⁺ enrichment column (R & D Systems, Inc., Minneapolis, Minn.). 5×10⁶ rat CD3⁺ cells were grown for three days in a 24 well plate, each well pre-coated with 1 μg/ml of anti-rat CD3⁺ antibody (BD Pharmingen, San Diego, Calif.), and treated with either conditioned medium from 293 cells expressing rat IL-21 or with conditioned medium from 293 cells that did not express IL-21, at a concentration of 0.1% or 0.02%. Supernatant was collected from cells after three days and tested for IL-10 production using an ELISA kit and following the manufacturer's instructions (R & D Systems, Inc., Minneapolis, Minn.).

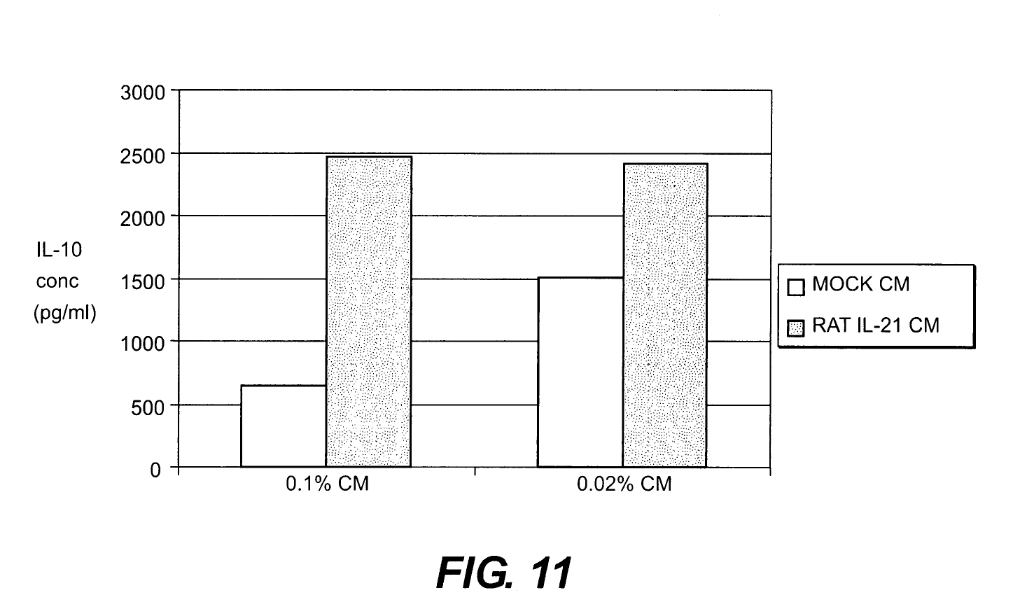

As depicted in FIG. 11, at both concentrations 0.02% and 0.1%, conditioned medium from 293 cells expressing rat IL-21 induced the rat CD3⁺ cells to secrete IL-10 into the supernatant at a much higher level compared to the cells that were treated with the conditioned medium from 293 cells that did not express rat IL-21.

### Example 8

### Cynomolius Monkey IL-21 Stimulates Proliferation of Cynomolaus Monkey White Blood Cells

Blood was collected from a cynomolgus monkey in Vacutainer CPT cell preparation tubes containing sodium citrate (Becton Dickinson, Franklin Lakes, N.J.). The blood was spun at room temperature at 1700 relative centrifugal force (RCF) for about 20 minutes. The plasma and interface layers were collected and washed twice in GIBCO® Hank's Buffer (Invitrogen). The red blood cells were lysed in milli-Q water for 1 minute and the white blood cells were washed in Hank's Buffer and resuspended in DME containing 10% FCS.

White blood cells were counted by trypan blue viable counts method using a hemocytometer and about 10,000 cells were plated onto a 96 well plate with each well pre-coated with 1 ng/ml of anti-human CD3⁺ antibody (clone SP34) (BD Pharmingen, San Jose, Calif.), and treated with either conditioned medium from cells expressing cynomolgus IL-21 or conditioned medium from 293 cells that did not express cynomolgus IL-21. The cells were grown for about three days in 10% CO₂ at 37° C. During the last five hours prior to harvesting the cells, the cells were labeled with 0.5 μCi methyl-3H thymidine/well (Amersham, Piscataway, N.J.). Cells were subsequently harvested using a Tomtec Mach III plate harvester (Wallac, Gaithersburg, Md.) and counted using a Perkin Elmer 1450 microbeta counter (Perkin Elmer, Wellesley, Mass.)

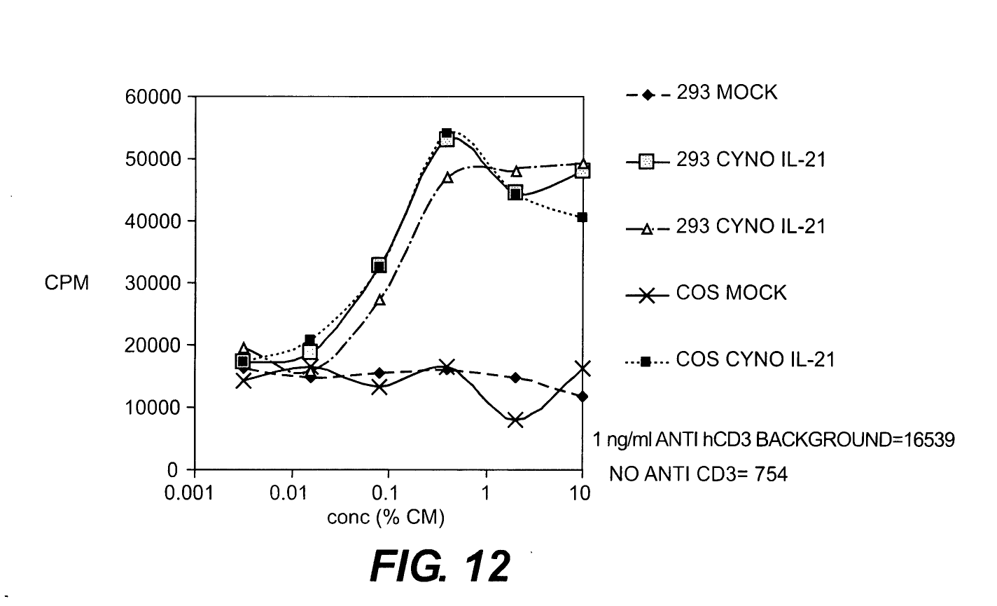

Cynomolgus monkey cells that were treated with conditioned medium from cells which expressed cynomolgus monkey IL-21 exhibited proliferation rates that were about two to six fold higher than the rate of proliferation of cells that were treated with conditioned medium from cells that did not express IL-21, as shown in FIG. 12.

### Example 9

### Cynomolgus Monkey IL-22 Stimulates GROa Secretion from HT29 Cells

Human colonic carcinoma cell line (HT29) cells, were plated in a 96 well plate (Corning Inc., Corning, N.Y.) at a concentration of 5×10⁴/well in DME medium containing 10% fetal bovine serum (FBS), 100 units/ml penicillin plus streptomycin and 2 mM glutamine. Approximately 24 hours later, medium was removed from HT29 cells and new medium with titrating amounts of human IL-22 (huIL-22), murine IL-22 (muIL-22), rat IL-22 (R & D Systems, Inc., Minneapolis, Minn.) or cynomolgus IL-22 (cyno IL-22) was added to the cells in the 96 well plate.

HT29 cells were incubated for about 48 hours at 37° C. and at 5% CO₂, medium was collected and secreted GROa was measured using Human GROa Immunoassay kit (R&D Systems, Minneapolis, Minn.), according to the manufacturer's instructions.

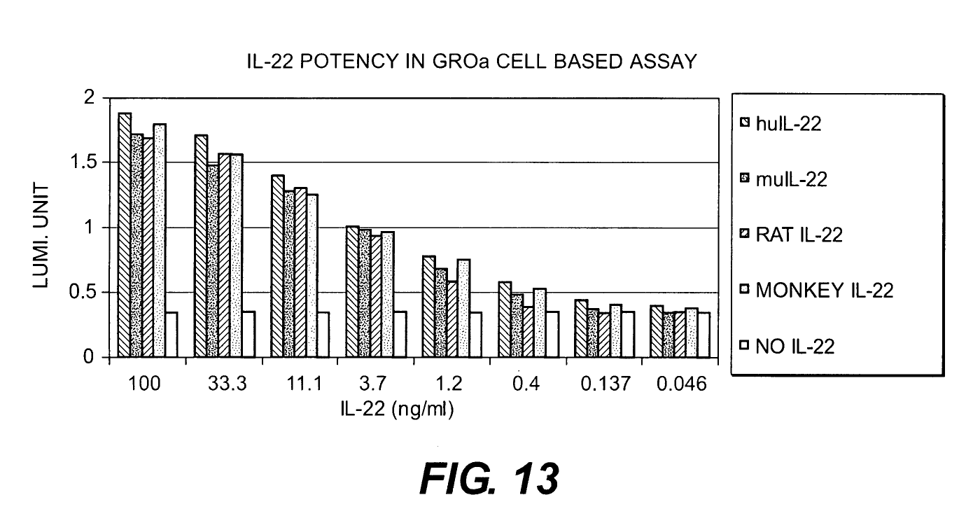

As depicted in FIG. 13, the cynomolgus IL-22 (cyno IL-22) stimulated GROa secretion from HT29 in amounts similar to the amounts secreted in the presence of human IL-22, rat IL-22 (R & D Systems, Inc., Minneapolis, Minn.) and murine IL-22.

### Example 10

### Cynomolaus IL-22 Induces Proliferation of BaF3 Cells Expressing Both the Human IL-22 Receptor and the Human IL-10 Receptor

BaF3 cells expressing both human IL-22 receptor (hIL-22R) and human IL-10 receptor (hIL-10R2) were generated by serial retroviral transduction of BaF3 cells with constructs expressing hIL-22R with green fluorescent protein (GFP) as a reporter and hIL-10R2 with yellow fluorescent protein (YFP) as a reporter. BaF3 cells expressing both hIL-22R and hIL-10R2 were sorted and collected using a fluorescent activated cell sorter (FACS).

BaF3 cells expressing hIL-22R and hIL-10R2 were maintained in Roswell Park Memorial Institute (RPMI) medium supplemented with 10% FBS, 2 mM glutamine, 100 units/ml penicillin plus streptomycin and 10 M HEPES. Additionally, 10 units/ml murine IL-3 (mIL3) was added to facilitate growth and proliferation when passaging the cells.

In order to evaluate proliferation of the BaF3 cells, they were spun down when they were in the logarithmic phase of growth, resuspended in RPMI medium at a concentration of approximately 10⁶ cells/ml and washed once with an equal volume of RPMI medium. A cell suspension of about 10⁵ cells/ml was made and 50 μl of the cell suspension was aliquoted into each well of a sterile flat-bottomed white 96 well plate (Thermo Labsystems, Franklin, Mass.).

Titrating amounts of human IL-22 (huIL-22), murine IL-22 (mu-IL22), rat IL-22 (R & D Systems, Inc., Minneapolis, Minn.) or cynomolgus IL-22 (cyno-IL-22) were added to the cells at 50 μl/well and the cells were incubated at 37° C. in a 5% CO₂ humidified incubator. After approximately 48 hours, reconstituted CellTiter Glo™ reagent (Promega, Madison, Wis.) in the amount of 100 μl was added to each well and luminescence was measured using an Envision™ plate reader (Perkin Elmer, Wellesley, Mass.).

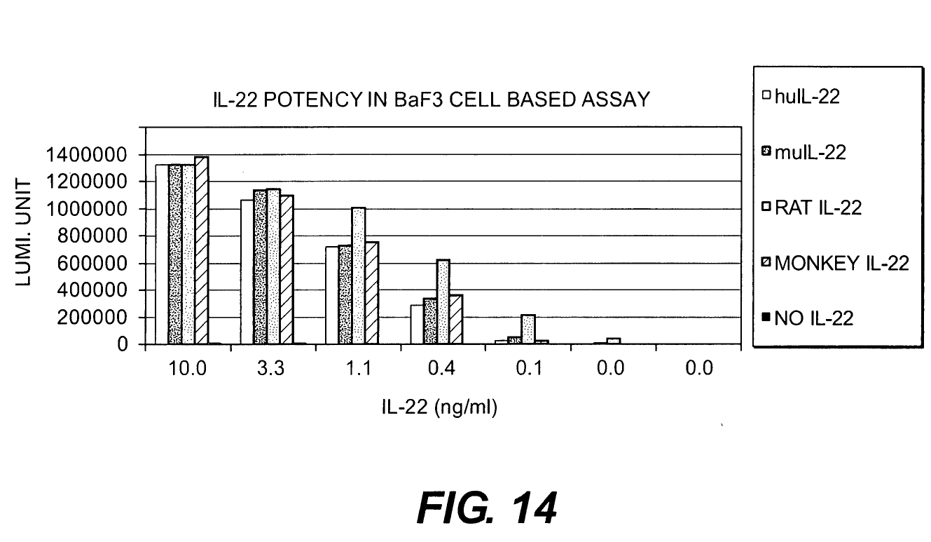

As shown in FIG. 14, increasing concentration of each of the cynomolgus IL-22 (cyno IL-22), human IL-22 (hu-IL-22), murine IL-22 (muIL-22) and rat IL-22 (R & D Systems, Inc., Minneapolis, Minn.) resulted in a corresponding increase in the proliferation of BaF3 cells, as measured by luminescence.

### Example 11

### Cloning of Rabbit Monkey IL-22 cDNA

The cDNA encoding rabbit IL-22 was PCR amplified from a cDNA pool derived from rabbit spleen, lung and testis. (Seegene, Inc., Seoul, South Korea).

The following PCR primers were used for amplification, 5′-CACCATGGCTGCCCTGCAGAGTCTG-3′ (SEQ ID NO:41) and 5′-TTACTCATTTTCCAGCTTTGCTC-3′ (SEQ ID NO:42) were derived from the Broad Institute contig 211103 of the rabbit genome sequencing project that had similarity to mouse IL-22 Genbank® Accession No. NM_{—}016971 and were used in a PCR reaction with the Advantage2 polymerase (Clontech, Palo Alto, Calif.) according to the manufacture's protocols. The purified PCR product of 586 bp was cloned into the pCRBluntII-topo plasmid (Invitrogen). Multiple independently derived PCR products were sequenced to arrive at the consensus sequence shown in SEQ ID NO:39.

The rabbit IL-22 cDNA was subsequently amplified by PCR using nested primers including the signal peptide of honeybee prepromelittin and His6x-FLAG tags (SEQ ID NO: 46) and Gatewaye attB sites using the following primers: 5′-GGGGACAGTTTGTACAAAAAGCAGGCTCCACCATGAAATTCTTAGTCA CGTTGCCCTTGTTTTTATGGTCGTGTACATTTCTTACATCTATGCG-3′ (SEQ ID NO:31), which contains the Gateway® attB1 site and the honeybee prepromelittin signal peptide sequence; 5′-ATTTCTTACATCTATGCGGGTAGCGGCCACCATCACCA CCACCAC-3′ (SEQ ID NO:32), which contains a 2×(glycine-serine) linker (SEQ ID NO: 45), a His6x tag (SEQ ID NO: 46), and a partial overlapping sequence to the signal sequence; 5′-CACCATCACCACCA CCACGGTAGCGGCGACTACAAAGACGATGAC-3′ (SEQ ID NO:33), which contains a His 6x tag (SEQ ID NO: 46), another 2×(glycine-serine) linker (SEQ ID NO: 45), and a partial overlapping FLAG tag sequence; 5′-GACTACAAAGACGATGACGACAAGCTGCCCATCAGCTCCCACTGC-3′ (SEQ ID NO:43), which contains a FLAG tag sequence and a rabbit IL-22 specific sequence; 5′-ACTTTGTACAGAAAGCTGGGTTTAACTCATTTTCCAGCTTTGC-3′ (SEQ ID NO:44), which contains a rabbit IL-22 specific sequence, a stop codon, and a partial overlapping Gateway® attB2 site; and 5′-GGGGACCACTTTGTACAAGAAAGCTGGGT-3′ (SEQ ID NO:36), which contains a full Gateway® attB2 site, and cloned into the Gateway® cloning platform into the pDONR221 plasmid using BP CLONASE™ and ultimately into the pSMED2 plasmid using LR CLONASE™.

### Example 12

### Rabbit IL-22 Induces Proliferation of BaF3 Cells Expressing both the Human IL-22 Receptor and the Human IL-10 Receptor

BaF3 cells expressing both human IL-22 receptor (hIL-22R) and human IL-10 receptor (hIL-10R2) were generated by serial retroviral transduction of BaF3 cells with constructs expressing hIL-22R with green fluorescent protein (GFP) as a reporter and hIL-10R2 with yellow fluorescent protein (YFP) as a reporter. BaF3 cells expressing both hIL-22R and hIL-10R2 were sorted and collected using a fluorescent activated cell sorter (FACS).

BaF3 cells expressing hIL-22R and hIL-10R2 were maintained in Roswell Park Memorial Institute (RPMI) medium supplemented with 10% FBS, 2 mM glutamine, 100 units/ml penicillin plus streptomycin and 10 M HEPES. Additionally, 10 units/ml murine IL-3 (mIL3) was added to facilitate growth and proliferation when passaging the cells.

In order to evaluate proliferation of the BaF3 cells, they were spun down when they were in the logarithmic phase of growth, resuspended in RPMI medium at a concentration of approximately 10⁶ cells/ml and washed once with an equal volume of RPMI medium. A cell suspension of about 10⁵ cells/ml was made and 50 μl of the cell suspension was aliquoted into each well of a sterile flat-bottomed white 96 well plate (Thermo Labsystems, Franklin, Mass.).

Titrating amounts of human IL-22 (huIL-22), and rabbit IL-22 were added to the cells at 50 μl/well and the cells were incubated at 37° C. in a 5% CO₂ humidified incubator. After approximately 48 hours, reconstituted CellTiter Glo™ reagent (Promega, Madison, Wis.) in the amount of 100 μl was added to each well and luminescence was measured using an Envision™ plate reader (Perkin Elmer, Wellesley, Mass.).

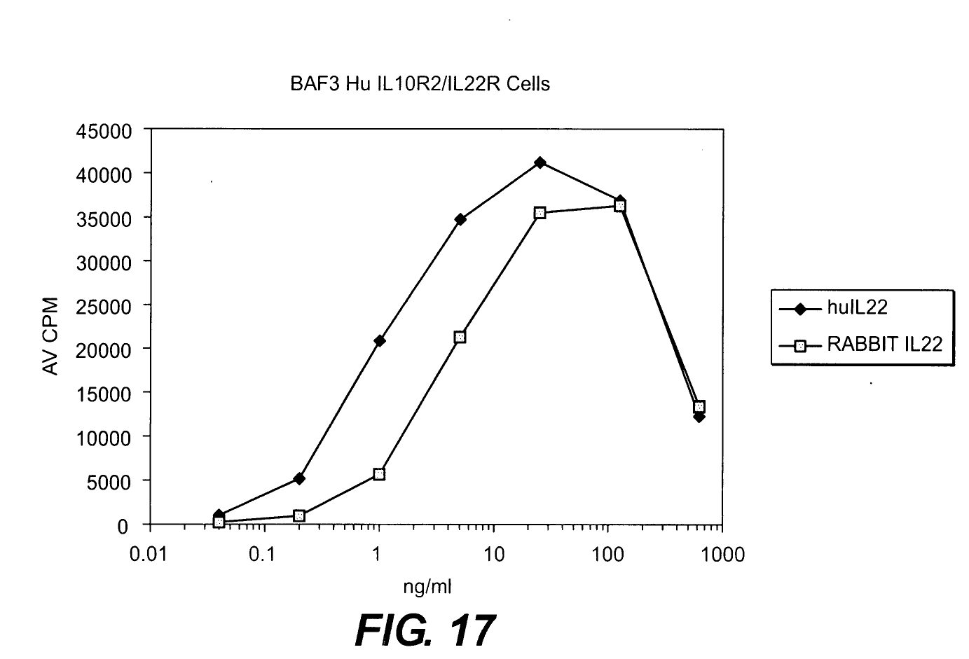

As shown in FIG. 17, increasing concentration of each of the rabbit IL-22 and human IL-22 (hu-IL-22) resulted in a corresponding increase in the proliferation of BaF3 cells, as measured by luminescence.

The specification is most thoroughly understood in light of the teachings of the references cited within the specification which are hereby incorporated by reference. The embodiments within the specification provide an illustration of embodiments encompassed by this disclosure and should not be construed to limit its scope. The skilled artisan readily recognizes that many other embodiments are encompassed by this specification. All publications and patents cited are incorporated by reference in their entirety. To the extent the material incorporated by reference contradicts or is inconsistent with the present specification, the present specification will supercede any such material. The citation of any references herein is not an admission that such references are prior art.

Unless otherwise indicated, all numbers expressing quantities of ingredients, treatment conditions, and so forth used in the specification, including claims, are to be understood as being modified in all instances by the term “about.” Accordingly, unless otherwise indicated to the contrary, the numerical parameters are approximations and may very depending upon the desired properties sought to be obtained. Unless otherwise indicated, the term “at least” preceding a series of elements is to be understood to refer to every element in the series. Those skilled in the art will recognize, or be able to ascertain using no more than routine experimentation, many equivalents to the specific embodiments described herein. Such equivalents are intended to be encompassed by the following claims.

What is claimed is:

1. An isolated nucleic acid molecule comprising:
(a) a nucleotide sequence set forth in SEQ ID NO:1;
(b) a nucleotide sequence at least 95% identical to a nucleotide sequence comprising nt 1 to 438 of SEQ ID NO:1 or nt 52 to 438 of SEQ ID NO:1 and encoding a protein having IL-21 activity;
(c) a nucleotide sequence encoding the amino acid sequence set forth in SEQ ID NO:2 or encoding aa 18 to 146 of SEQ ID NO:2; or
(d) a nucleotide sequence set forth in SEQ ID NO:1 from nt 52 to nt 438.

2. A vector comprising an isolated nucleic acid molecule of claim 1.

3. A host cell transformed with the vector of claim 2.

4. An isolated polypeptide encoded by the nucleic acid molecule of claim 1.

5. An isolated polypeptide having IL-21 activity comprising:
(a) an amino acid sequence set forth in SEQ ID NO:2;
(b) an amino acid sequence at least 95% identical to the amino acid sequence set forth in SEQ ID NO:2; or
(c) an amino acid sequence set forth in SEQ ID NO:2 from aa 18 to aa 146.

6. An isolated nucleic acid molecule comprising:
(a) a nucleotide sequence set forth in SEQ ID NO:3;
(b) a nucleotide sequence at least 99% identical to a nucleotide sequence comprising nt 1 to 486 of SEQ ID NO:3 or nt 88 to 486 of SEQ ID NO:3 and encoding a protein having IL-21 activity;
(c) a nucleotide sequence encoding the amino acid sequence set forth in SEQ ID NO:4 or encoding aa 30 to 162 of SEQ ID NO:4; or
(d) a nucleotide sequence set forth in SEQ ID NO:3 from nt 88 to nt 486.

7. A vector comprising the nucleic acid molecule of claim 6.

8. A host cell comprising the vector of claim 7.

9. An isolated polypeptide encoded by the nucleic acid molecule of claim 5.

10. An isolated polypeptide having IL-21 activity and comprising:
(a) an amino acid sequence set forth in SEQ ID NO:4;
(b) an amino acid sequence at least 97% identical to the amino acid sequence set forth in SEQ ID NO:4; or
(c) an amino acid sequence set forth in SEQ ID NO:4 from aa 30 to aa 162.

11. A method of producing a protein having IL-21 activity comprising:
(a) culturing the host cell of claim 3 or 8 under conditions such that the protein having IL-21 activity is expressed; and
(b) recovering the protein.

12. A method of identifying a compound which modulates IL-21 activity comprising:
(a) contacting a polypeptide comprising an amino acid sequence set forth in SEQ ID NO:2, aa 18 to aa 146 of SEQ ID NO:2, SEQ ID NO:4 or aa 30 to aa 162 of SEQ ID NO:4 with a compound; and
(b) determining whether the compound modulates the IL-21 activity.

13. The method of claim 12, wherein the compound is an antagonist of IL-21 activity.

14. The method of claim 12, wherein the compound is an agonist of IL-21 activity.

15. A method of treating a condition associated with a decrease in IL-21 activity relative to the activity in absence of the condition comprising administering an effective amount of the IL-21 polypeptide of claims 4, 5, 9 or 10.

16. A method of treating a condition associated with an increase in IL-21 activity relative to the activity in the absence of the condition comprising administering a compound identified as an antagonist of IL-21 activity by the method of claim 12.

17. An isolated nucleic acid molecule comprising:
(a) a nucleotide sequence set forth in SEQ ID NO:5;
(b) a nucleotide sequence at least 95% identical to a nucleotide sequence comprising nt 1 to 537 of SEQ ID NO:5 or nt 100 to 537 of SEQ ID NO:5 and encoding a protein having IL-22 activity;
(c) a nucleotide sequence encoding the amino acid sequence set forth in SEQ ID NO:6 or encoding aa 34 to 179 of SEQ ID NO:6; or
(d) a nucleotide sequence set forth in SEQ ID NO:5 from nt 100 to nt 537.

18. A vector comprising an isolated nucleic acid molecule of claim 17.

19. A host cell transformed with the vector of claim 18.

20. An isolated polypeptide encoded by the nucleic acid molecule of claim 17.

21. An isolated polypeptide having IL-22 activity comprising:
(a) an amino acid sequence set forth in SEQ ID NO:6;
(b) an amino acid sequence at least 95% identical to the amino acid sequence set forth in SEQ ID NO:6; or
(c) an amino acid sequence set forth in SEQ ID NO:6 from aa 34 to aa 179.

22. An isolated nucleic acid molecule comprising:
(a) a nucleotide sequence set forth in SEQ ID NO:7;
(b) a nucleotide sequence at least 98% identical to a nucleotide sequence comprising nt 32 to 568 of SEQ ID NO:7 or nt 131 to 568 of SEQ ID NO:7 and encoding a protein having IL-22 activity;
(c) a nucleotide sequence encoding the amino acid sequence set forth in SEQ ID NO:8 or encoding aa 34 to 179 of SEQ ID NO:8; or
(d) a nucleotide sequence set forth in SEQ ID NO:7 from nt 131 to nt 568.

23. A vector comprising an isolated nucleic acid molecule of claim 22.

24. A host cell transformed with the vector of claim 23.

25. An isolated polypeptide encoded by the nucleic acid molecule of claim 22.

26. An isolated polypeptide having IL-22 activity comprising:
(a) an amino acid sequence set forth in SEQ ID NO:8;
(b) an amino acid sequence at least 95% identical to the amino acid sequence set forth in SEQ ID NO:8; or
(c) an amino acid sequence set forth in SEQ ID NO:8 from aa 34 to aa 179.

27. An isolated nucleic acid molecule comprising:
(a) a nucleotide sequence set forth in SEQ ID NO:39;
(b) a nucleotide sequence at least 95% identical to a nucleotide sequence comprising nt 1 to 561 of SEQ ID NO:39 or nt 100 to 561 of SEQ ID NO:39 and encoding a protein having IL-22 activity;
(c) a nucleotide sequence encoding the amino acid sequence set forth in SEQ ID NO:40 or encoding aa 34 to 187 of SEQ ID NO:40; or
(d) a nucleotide sequence set forth in SEQ ID NO:39 from nt 100 to nt 561.

28. A vector comprising an isolated nucleic acid molecule of claim 27.

29. A host cell transformed with the vector of claim 28.

30. An isolated polypeptide encoded by the nucleic acid molecule of claim 27.

31. An isolated polypeptide having IL-22 activity comprising:
(a) an amino acid sequence set forth in SEQ ID NO:40;
(b) an amino acid sequence at least 95% identical to the amino acid sequence set forth in SEQ ID NO:40; or
(c) an amino acid sequence set forth in SEQ ID NO:40 from aa 34 to aa 187.

32. A method of producing a protein having IL-22 activity comprising:
(a) culturing the host cell of claim 19, 24 or 29 under conditions such that the protein having IL-22 activity is expressed; and
(b) recovering the protein.

33. A method of identifying a compound which modulates an IL-22 activity comprising:
(a) contacting a polypeptide comprising an amino acid sequence set forth in SEQ ID NO:6, aa 34 to aa 179 of SEQ ID NO:6, SEQ ID NO:8, aa 34 to aa 179 of SEQ ID NO:8, SEQ ID NO:40, or aa 34 to aa 187 of SEQ ID NO:40 with a compound; and
(b) determining whether the compound modulates the IL-22 activity.

34. The method of claim 33, wherein the compound is an antagonist of IL-22 activity.

35. The method of claim 33, wherein the compound is an agonist of IL-22 activity.

36. A method of treating a condition associated with a decrease in IL-22 activity relative to the activity in absence of the condition comprising administering an effective amount of the IL-22 polypeptide of claims 20, 21, 25, 26, 30 or 31.

37. A method of treating a condition associated with an increase in IL-22 activity relative to the activity in absence of the condition comprising administering a compound identified as an antagonist of IL-22 activity by the method of claim 33.

38. The method of claim 12 or claim 33, wherein the compound is a small molecule.

39. The method of claim 12 or claim 33, wherein the compound is a peptide.

40. The method of claim 12 or claim 33, wherein the compound is an antibody.

41. An antibody which selectively binds to a polypeptide comprising an amino acid sequence set forth in SEQ ID NOs:2, 4, 6, 8 or 40.

42. The antibody of claim 41, wherein the antibody is a monoclonal antibody.

43. The antibody of claim 41, wherein the antibody is a polyclonal antibody.


SEQUENCE LISTING (46 sequences)

SEQ ID NO: 1 - DNA, 441 bp, Rattus sp.
atggagaggacccttgtctgtctgatactcatcttcttggggaccgtggcccataaatcaagcccccaaaggccagatcaccttctgattagacttcgtcaccttatggacattgttgaacagctgaaaatctatgagaatgacttggatcctgaacttctaacagctccacaagatgtaaaggggcaatgtgagcacgaagcttttgcctgttttcagaaagccaaactcaagccatcaaacactggaaacaataagacattcatcaacgacttgttggcacagctgaggaggaggctgcctgctaagaggacaggaaataaacagaggcacatggctaaatgcccttcctgtgatttgtatgagaaaaaaacacccaaagaattccttgaaagactaaaatggctccttcaaaagatgattcatcagcatctttcctag

SEQ ID NO: 2 - protein, 146 aa, Rattus sp.
MERTLVCLILIFLGTVAHKSSPQRPDHLLIRLRHLMDIVEQLKIYENDLDPELLTAPQDVKGQCEHEAFACFQKAKLKPSNTGNNKTFINDLLAQLRRRLPAKRTGNKQRHMAKCPSCDLYEKKTPKEFLERLKWLLQKMIHQHLS

SEQ ID NO: 3 - DNA, 489 bp, Macaca fascicularis
atgagatccagtcctggcaacatggagaggatagtcatctgtctgatggtcatcttcttggggacactggtccacaaatcaagctcccaaggtcaagatcgccacatgattagaatgcgtcaacttatagatattgttgatcagctgaaaaattatgtgaatgacttggaccctgaatttctgccagctccagaagatgtagagacaaactgtgagtggtcagctatttcctgttttcagaaggcccaactaaagtcagcaaatacaggaaacaatgaaaggataatcaatttatcaattaaaaagctgaagaggaaatcaccttccacaggtgcagagagaagacagaaacacagactaacatgcccttcatgtgattcttatgagaaaaaaccacccaaagaattcctagaaagattcaaatcacttctccaaaagatgattcatcagcatctgtcctctagaacacatggaagtgaagattcctga

SEQ ID NO: 4 - protein, 162 aa, Macaca fascicularis
MRSSPGNMERIVICLMVIFLGTLVHKSSSQGQDRHMIRMRQLIDIVDQLKNYVNDLDPEFLPAPEDVETNCEWSAISCFQKAQLKSANTGNNERIINLSIKKLKRKSPSTGAERRQKHRLTCPSCDSYEKKPPKEFLERFKSLLQKMIHQHLSSRTHGSEDS

SEQ ID NO: 5 - DNA, 537 bp, Rattus sp.
atgtctgtcctgaggaaatctatgagcttttcccttatggggactttggccgccagctgcctgcttctcgttgctctgtgggctcagaaggcagatgctctgcccatcaactcccaatgcaaacttgaggcagccaacttccagcagccatacatcgtcaaccgcacctttatgctggccaaggaggccagccttgcagataacaacacagatgtcaggctcatcggggaggaactgttccgaggagtcaaagccaaggatcaatgctacttgatgaagcaggtgctcaacttcacccttgaagatgttctgctcccccagtcagacagattccagccttacatgcaggaggtggtgcctttcctgaccaagctcagcagtcacctcagcccctgtcacatcagcggtgatgaccagaacatccagaagaacgtaagacagctgaaggaaacagtgcaaaagcttggagagagtggagagattaaagcgatcggggaactggatctgctgtttatgtccctgagaaacgcttgcgtc

SEQ ID NO: 6 - protein, 179 aa, Rattus sp.
MSVLRKSMSFSLMGTLAASCLLLVALWAQKADALPINSQCKLEAANFQQPYIVNRTFMLAKEASLADNNTDVRLIGEELFRGVKAKDQCYLMKQVLNFTLEDVLLPQSDRFQPYMQEVVPFLTKLSSHLSPCHISGDDQNIQKNVRQLKETVQKLGESGEIKAIGELDLLFMSLRNACV

SEQ ID NO: 7 - DNA, 653 bp, Macaca fascicularis
tcaccaattgctcgagtaagagttgtctgcaatggccgccctgcagaaatctgtgagccctttccttatggggactctggccaccagctgcctccttctcttggccctctgggtgcagggaggagcagctgcgcccgtcagctcccactgcaggcttgacaagtccaacttccagcagccctatatcaccaaccgcaccttcatgctggctaaggaggctagctcggctgataacaacacagacgttcgtctcattggggaaaaactgttccgcggagtcagtatgagtgagcgctgctatctgatgaagcaggtgctgaacttcacccttgaagaagtgctgctccctcaatcggataggttccagccttatatgcaggaggtggtgcccttcctggccaggctcagcaacagtctaagcacatgtcacattgaaggtgatgacctgcatatccagaagaatgtccaaaagctgaaggacacagtgaaaaagcttggagagagtggagagatcaaagcaattggagaactggatttgctgtttatgtctctgagaaatgcctgcatttgaccagaacaaagctgaaaaacgaataactaaccctttttccctgatagaaataacagttagatcccccaaagcgatttttttt

SEQ ID NO: 8 - protein, 179 aa, Macaca fascicularis
MAALQKSVSPFLMGTLATSCLLLLALWVQGGAAAPVSSHCRLDKSNFQQPYITNRTFMLAKEASSADNNTDVRLIGEKLFRGVSMSERCYLMKQVLNFTLEEVLLPQSDRFQPYMQEVVPFLARLSNSLSTCHIEGDDLHIQKNVQKLKDTVKKLGESGEIKAIGELDLLFMSLRNACI

SEQ ID NO: 9 - DNA, 441 bp, Mus musculus
atggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcatcgacctcgtggcccagctgaggaggaggctgcctgccaggaggggaggaaagaaacagaagcacatagctaaatgcccttcctgtgattcgtatgagaaaagaacacccaaagaattcctagaaagactaaaatggctccttcaaaagatgattcatcagcatctctcctag

SEQ ID NO: 10 - protein, 146 aa, Mus musculus [sequence omitted]

SEQ ID NO: 11 - DNA, 489 bp, Homo sapiens
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

SEQ ID NO: 12 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 13 - DNA, 537 bp, Mus musculus
atggctgtcctgcagaaatctatgagtttttcccttatggggactttggccgccagctgcctgcttctcattgccctgtgggcccaggaggcaaatgcgctgcccgtcaacacccggtgcaagcttgaggtgtccaacttccagcagccgtacatcgtcaaccgcacctttatgctggccaaggaggccagccttgcagataacaacacagacgtccggctcatcggggagaaactgttccgaggagtcagtgctaaagatcagtgctacctgatgaagcaggtgctcaacttcaccctggaagacgttctgctcccccagtcagacaggttccagccctacatgcaggaggtggtacctttcctgaccaaactcagcaatcagctcagctcctgtcacatcagcggtgacgaccagaacatccagaagaatgtcagaaggctgaaggagacagtgaaaaagcttggagagagtggagagatcaaggcgattggggaactggacctgctgtttatgtctctgagaaatgcttgcgtc

SEQ ID NO: 14 - protein, 179 aa, Mus musculus
MAVLQKSMSFSLMGTLAASCLLLIALWAQEANALPVNTRCKLEVSNFQQPYIVNRTFMLAKEASLADNNTDVRLIGEKLFRGVSAKDQCYLMKQVLNFTLEDVLLPQSDRFQPYMQEVVPFLTKLSNQLSSCHISGDDQNIQKNVRRLKETVKKLGESGEIKAIGELDLLFMSLRNACV

SEQ ID NO: 15 - DNA, 653 bp, Homo sapiens
tcaccagttgctcgagttagaattgtctgcaatggccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcatttgaccagagcaaagctgaaaaatgaataactaaccccctttccctgctagaaataacaattagatgccccaaagcgatttttttt

SEQ ID NO: 16 - protein, 179 aa, Homo sapiens [sequence omitted]

SEQ ID NO: 17 - DNA, 44 bp, Artificial Sequence
  Description of Artificial Sequence Synthetic primer
gtacaaaaaagcaggctccaccatggagaggacccttgtctgtc

SEQ ID NO: 18 - DNA, 44 bp, Artificial Sequence
  Description of Artificial Sequence Synthetic primer
actttgtacaagaaagctgggtctaggaaagatgctgatgaatc

SEQ ID NO: 19 - DNA, 24 bp, Artificial Sequence
  Description of Artificial Sequence Synthetic primer
gctgaagtgaaaacgagaccaagg

SEQ ID NO: 20 - DNA, 26 bp, Artificial Sequence
  Description of Artificial Sequence Synthetic primer
gaatacaaagaaatgactttcactac

SEQ ID NO: 21 - DNA, 25 bp, Artificial Sequence
  Description of Artificial Sequence Synthetic primer
aaacgagaccaaggtctagctctac

SEQ ID NO: 22 - DNA, 24 bp, Artificial Sequence
  Description of Artificial Sequence Synthetic primer
attagagtatgtaacatagtgtcc

SEQ ID NO: 23 - DNA, 55 bp, Artificial Sequence
  Description of Artificial Sequence Synthetic primer
ggggacaagtttgtacaaaaaagcaggctatatgagatccagtcctggcaacatg

SEQ ID NO: 24 - DNA, 60 bp, Artificial Sequence
  Description of Artificial Sequence Synthetic primer
ggggaccactttgtacaagaaagctgggtatcactaggaatcttcacttccgtgtgttct

SEQ ID NO: 25 - DNA, 31 bp, Artificial Sequence
  Description of Artificial Sequence Synthetic primer
caccatgtctgtcctgaggaaatctatgagc

SEQ ID NO: 26 - DNA, 53 bp, Artificial Sequence
  Description of Artificial Sequence Synthetic primer
gtggtggtggtgatggtgggaccccgaccctgcgacgcaagcgtttctcaggg

SEQ ID NO: 27 - DNA, 54 bp, Artificial Sequence
  Description of Artificial Sequence Synthetic primer
ggggacaagtttgtacaaaaaagcaggctccaccatgtctgtcctgaggaaatc

SEQ ID NO: 28 - DNA, 74 bp, Artificial Sequence
  Description of Artificial Sequence Synthetic primer
ggggaccactttgtacaagaaagctgggttcacttgtcgtcatcgtctttgtagtcgtggtggtggtgatggtg

SEQ ID NO: 29 - DNA, 20 bp, Artificial Sequence
  Description of Artificial Sequence Synthetic primer
accaggttctccttccccag

SEQ ID NO: 30 - DNA, 21 bp, Artificial Sequence
  Description of Artificial Sequence Synthetic primer
ggcttcccatcttccttttgg

SEQ ID NO: 31 - DNA, 97 bp, Artificial Sequence
  Description of Artificial Sequence Synthetic primer
ggggacaagtttgtacaaaaaagcaggctccaccatgaaattcttagtcaacgttgcccttgtttttatggtcgtgtacatttcttacatctatgcg

SEQ ID NO: 32 - DNA, 45 bp, Artificial Sequence
  Description of Artificial Sequence Synthetic primer
atttcttacatctatgcgggtagcggccaccatcaccaccaccac

SEQ ID NO: 33 - DNA, 45 bp, Artificial Sequence
  Description of Artificial Sequence Synthetic primer
caccatcaccaccaccacggtagcggcgactacaaagacgatgac

SEQ ID NO: 34 - DNA, 44 bp, Artificial Sequence
  Description of Artificial Sequence Synthetic primer
gactacaaagacgatgacgacaaggcgcccgtcagctcccactg

SEQ ID NO: 35 - DNA, 44 bp, Artificial Sequence
  Description of Artificial Sequence Synthetic primer
ctttgtacaagaaagctgggttcaaatgcaggcatttctcagag

SEQ ID NO: 36 - DNA, 29 bp, Artificial Sequence
  Description of Artificial Sequence Synthetic primer
ggggaccactttgtacaagaaagctgggt

SEQ ID NO: 37 - DNA, 37 bp, Artificial Sequence
  Description of Artificial Sequence Synthetic primer
ggggacaagtttgtacaaaaaagcaggctccaccatg

SEQ ID NO: 38 - DNA, 29 bp, Artificial Sequence
  Description of Artificial Sequence Synthetic primer
ggggaccactttgtacaagaaagctgggt

SEQ ID NO: 39 - DNA, 559 bp, Unknown
  Description of Unknown Rabbit cDNA sequence
atggctgccctgcagaagtctgtgatctcgttcctgatggggactctgggagccagcggcctcctgctcatggccctgatggcacacggaggagctgcgctgcccatcagctcccactgcaggctggacgagtccaacttcacttcctatatcaccaacctcaccttcatgctggctaaggaggccaattcggcagataacaacacagatgttcgtcttattgggaatgagctgttccatggagtcaatgagaaagaccactgctatctgctgaagcacgtgctcaactttacccttgaagaagtgctgttccccgagtctgatagattccagccctacatgcagcaggtgctgtccttcctggccaagcttagcaacaatctcagccaatgtcatattgaaggcgatgaccagcatatccagcggaatgtgcagcagctgaaggacacagtgaagcagcttggagagagtggagagatcaaagcaattggagaactggatctgctgtttatggccctgagaactgcctgcgtttcaccggagcaaagctggaaaatga

SEQ ID NO: 40 - protein, 178 aa, Unknown
  Description of Unknown Rabbit protein sequence
MAALQKSVISFLMGTLGASGLLLMALMAHGGAALPISSHCRLDESNFTSYITNLTFMLAKEANSADNNTDVRLIGNELFHGVNEKDHCYLLKHVLNFTLEEVLFPESDRFQPYMQQVLSFLAKLSNNLSQCHIEGDDQHIQRNVQQLKDTVKQLGESGEIKAIGELDLLFMALRTACV

SEQ ID NO: 41 - DNA, 26 bp, Artificial Sequence
  Description of Artificial Sequence Synthetic primer
caccatggctgccctgcagaagtctg

SEQ ID NO: 42 - DNA, 24 bp, Artificial Sequence
  Description of Artificial Sequence Synthetic primer
ttaactcattttccagctttgctc

SEQ ID NO: 43 - DNA, 45 bp, Artificial Sequence
  Description of Artificial Sequence Synthetic primer
gactacaaagacgatgacgacaagctgcccatcagctcccactgc

SEQ ID NO: 44 - DNA, 44 bp, Artificial Sequence
  Description of Artificial Sequence Synthetic primer
actttgtacaagaaagctgggtttaactcattttccagctttgc

SEQ ID NO: 45 - protein, 4 aa, Artificial Sequence
  Description of Artificial Sequence Synthetic peptide
GSGS

SEQ ID NO: 46 - protein, 6 aa, Artificial Sequence
  Description of Artificial Sequence Synthetic 6xHis tag
HHHHHH